(19) United States
(12) Patent Application Publication
(10) Pub. No.: US 2024/0044826 A1
(43) Pub. Date: Feb. 8, 2024

(54) METABOLIC VULNERABILITY ANALYZED BY NMR

(71) Applicant: LipoScience, Inc., Morrisville, NC (US)

(72) Inventor: James D. Otvos, Apex, NC (US)

(21) Appl. No.: 18/366,599

(22) Filed: Aug. 7, 2023

Related U.S. Application Data
(60) Provisional application No. 63/395,589, filed on Aug. 5, 2022.

(51) Int. Cl. G01N 24/08

(52) U.S. Cl. CPC G01N 24/08; G01N 2800/56

(57) ABSTRACT

Disclosed are methods and systems for evaluating various health markers to distinguish various health risks. In one embodiment, the method comprises evaluating biomarkers to determine a relative risk of premature all-cause mortality in metabolic malnutrition-inflammation syndrome (MMIS). A method may include obtaining NMR-derived measurements of GlycA, S-HDLP, branched chain amino acids (BCAAs), and citrate from a subject's sample.

### RELATED PATENT APPLICATIONS

This patent application claims the benefit of U.S. provisional patent application No. 63/395,589 filed on Aug. 5, 2022, entitled METABOLIC VULNERABILITY ANALYZED BY NMR. The entire content of the foregoing application is incorporated herein by reference, including all text, tables, and drawings.

### FIELD

The present disclosure relates generally to analysis of samples. The disclosure may be particularly suitable for Nuclear Magnetic Resonance (NMR) analysis of samples.

### BACKGROUND

There is a current interest among healthcare professionals and patients for techniques that can evaluate the relative risk of mortality in patients afflicted with certain disease states. While some disease states may impart a higher risk of mortality, at times, it can be a combination of one or more disease states that lead to an increased risk of mortality. Risk of mortality may not be limited only to one disease state or a combination thereof, however. Increased risk of mortality may be attributed to various factors that can be defined as risk factors herein. Risk factors can include cholesterol levels, comorbidities, familial health history (e.g., diabetes mellitus, cancer, hypertension), weight, age, and other factors that may affect health. Thereby, health markers, further referred to as biomarkers, that may correlate to certain risk factors can be identified to aid predictive models in assessing increased risk of mortality for a population. Many risk factors may be unpredictable in assessing risk of mortality and may, at times, not correlate well with increased risk of mortality. For example, a first person who smokes cigarettes can be at higher risk for lung cancer, and thereby mortality, than a second person who does not smoke, yet the second person may be at higher risk for mortality for a separate reason. Consequently, all-cause mortality is used as a term that further indicates the mortality of a population, regardless of cause or risk. Interest of identifying certain biomarkers to aid in predicting risk and evaluating all-cause mortality has become warranted.

While many disease states may impart a risk of mortality, of particular interest is the syndrome referred to as Malnutrition-Inflammation Complex Syndrome (MICS). MICS can be associated with accelerated atherosclerosis and may portend mortality. MICS can also be defined as the malnutrition-inflammation-associated muscle decay. In MICS, an individual may or may not display proper risk factors that would likely lead a clinician to begin a therapeutic course. Thus, there is a need to analyze biomarkers that may be associated with all-cause mortality and/or MICS.

### SUMMARY

Embodiments of the disclosure include methods and systems that can evaluate a person's relative risk of premature all-cause mortality and/or provide mortality risk stratification by evaluating NMR spectra of a biological specimen from a patient sample. A biological specimen from a patient sample may include, at least, blood, serum, saliva, urine, and sputum from which one of the biomarkers may be extracted for NMR examination. In an embodiment, the analyte of interest can include a biomarker associated with MICS. In an embodiment, the analytes are analyzed using NMR. NMR, particularly proton NMR, has the capacity to detect protons of a sample relative to a structure of a compound in the sample. In essence, NMR can allow for a user to predict a particular structure of a compound or otherwise, using an internal standard, detect a particular analyte of interest.

It can be postulated that NMR biomarkers reflect or can be manifestations of the metabolic derangements underlying protein-energy wasting or metabolic malnutrition as opposed to the undernutrition aspects of malnutrition and its clinical diagnostic phenotypes. Thus, in many cases MICS can be analogous for MMIS (Metabolic Malnutrition-Inflammation Syndrome). Many Biomarkers can be analyzed for increased understanding of MICS/MMIS. For example, biomarkers that can be evaluated may include low serum albumin, C-reactive protein, GlycA, S-HDLP, citrate, and branched-chain amino acids (BCAAs) that include valine, leucine, and isoleucine. These biomarkers can be parameters that can aid in establishing the boundaries of MICS in a patient where low serum albumin refers to blood-bound globular proteins that are made by the liver and can aid in retaining volume in the blood stream. Thus, a low level of serum albumin may correlate with malnutrition and potentially liver failure. More specifically, simultaneously measuring GlycA and S-HDLP with small molecule metabolites such as citrate and the BCAAs can define the parameters of metabolic malnutrition-inflammation syndrome (MMIS). C-reactive protein is a protein also produced by the liver that is generated in response to high inflammation within the body. GlycA is a biomarker that is also associated with systemic inflammation. S-HDLP can be defined as small-high density lipoprotein particles that are primarily synthesized in the liver. Citrate is an organic molecule that is generated in response to respiration at the level of the Kreb's cycle and can have implications in altering the pH of various fluids including serum. Finally, valine, leucine, and isoleucine (e.g., branched-chain amino acids, BCAA) are part of a larger group of nine amino acids that can be referred to as essential amino acids. The body does not produce the nine essential amino acids meaning dietary habits may ultimately play a role in mitigating MICS.

In one embodiment, a user may obtain a sample of blood, serum, or plasma from a subject for sampling or analyzing by nuclear magnetic resonance (NMR) spectrometry. Following sample collection and analysis, deconvolution of the NMR spectrum may be performed. In an embodiment, the deconvolution comprises determining the presence and the area (i.e., corresponding to concentration) of a signal that corresponds to a certain biomarker. For example, the structure of GlycA can be difficult to extrapolate from a proton NMR spectrum, particularly in a case where one or more biomarkers are overlapping in signal, yet for ease, considering GlycA maintains a consistent moiety within its structure, a proton location of the consistent moiety can be sought after and used computationally to account for the molecule as a whole. Alternatively, a consistent, individual or set of protons for a second biomarker can be used in identifying the presence of one or more biomarkers when overlap may not occur. While proton spectra can overlap when all present biomarkers are simultaneously detected, a known, consistent location for a moiety within at least one biomarker may be readily detected, allowing a user to extrapolate spectra and detect the presence of all the biomarkers contemporaneously. The term location refers to the x-axis of a proton NMR spectra read in units of parts per million ppm).

In one embodiment of the disclosure, a method for determining the mortality risk associated with MVX can be used, where MVX is known as the metabolic vulnerability index and where the MVX score can correlate to mortality risk. Additionally, one embodiment of the disclosure may include a method for calculating MVX where MVX can be derived from the detection of the biomarkers low serum albumin, C-reactive protein, GlycA, S-HDLP, citrate, valine, leucine, and isoleucine, and more particularly the simultaneous measurement of GlycA, S-HDLP, citrate, valine, leucine, and isoleucine exclusive of low serum albumin and C-reactive protein. Another embodiment of the present disclosure may include a method for calculating MVX where MVX can be derived from the detection of biomarkers that include GlycA, S-HDLP, citrate, valine, leucine, and isoleucine and more particularly the simultaneous measurements of the six biomarkers GlycA, S-HDLP, citrate, valine, leucine, and isoleucine. Further, a multi-marker score can range from 1-100 and can be calculated using citrate and the three BCAAs to generate an index titled Metabolic Malnutrition Index (MMX). Similarly, GlycA and S-HDLP can be used to generate an index referred to as Inflammation Vulnerability Index (IVX). When combining IVX and MMX, MVX, a composite MICS (or MMIS) multi-marker, may be calculated. Combining biomarkers of malnutrition and inflammation, may yield the total, combined markers of Malunion-Inflammation Composite Syndrome (MICS) referred to as MVX. MVX may have a strong correlation to mortality rates when evaluated over the course of a five-year period from one or more cohorts. Thus, allowing a researcher to predict all-cause mortality rates for MICS based on calculated values of IVX and MMX as generated from proton NMR spectroscopy.

Embodiments of the disclosure may include collecting demographical data from at least one or more cohorts of a population. The cohorts may include a cohort from Catheterization Genetics, known as CATHGEN, and may include a cohort from Intermountain Heart Collaborative Study for a combination of patients that may total to over eight thousand participants. Having a large population of patients can allow for increased representation when generating statistical conclusions.

Further features, advantages, and details of the present disclosure will be appreciated by those of ordinary skill in the art from a reading of the figures and the detailed description of the preferred embodiments that follow, such description being merely illustrative of the present disclosure. Features described with respect with one embodiment can be incorporated with other embodiments although not specifically discussed therewith. That is, it is noted that aspects of the disclosure described with respect to one embodiment, may be incorporated in a different embodiment although not specifically described relative thereto. That is, all embodiments and/or features of any embodiment can be combined in any way and/or combination. The foregoing and other aspects of the present disclosure are explained in detail in the specification set forth below.

### BRIEF DESCRIPTION OF THE FIGURES

FIG. 1 is a table showing the baseline characteristics of CATHGEN and Intermountain Heart cardiac catheterization cohorts in accordance with one embodiment of the disclosure.

FIG. 2 is a table showing associations of IVX, MMX and MVX with a 5-year Mortality in CATHGEN and Intermountain Heart cohorts in accordance with one embodiment of the disclosure.

FIG. 3 is a table showing associations of MVX with a 5-year mortality in CATHGEN patient subgroups in accordance with one embodiment of the disclosure.

FIG. 4 is a plot that shows four graphs that describe the relative contributions of a given variable to the prediction of all-cause mortality and non-fatal MI in CATHGEN, calculated as percent of the total chi-square of the cox regression model in accordance with one embodiment of the disclosure.

FIG. 5 is a graph showing the 5-year age-adjusted mortality of baseline IVX and MMX scores in CATHGEN in accordance with one embodiment of the disclosure. The number of subjects in each subgroup is given in parentheses. The synergy (interaction) between IVX and MMX is reflected by the much greater influence each has on mortality risk when levels of the other are higher versus lower.

FIG. 6 is a graph showing the cumulative mortality incidence by baseline MVX score in the CATHGEN cohort in accordance with one embodiment of the disclosure.

FIG. 7 is a schematic depicting the various considerations that may apply to disease and mortality prevention in accordance with one embodiment of the disclosure.

FIG. 8 is a table showing the calculations of sex-specific IVX, MMX, and MVX scores in accordance with one embodiment of the disclosure.

FIG. 9 is a table showing spearman correlations in CATHGEN and Intermountain Heart cohorts in accordance with one embodiment of the disclosure.

FIG. 10 is a table showing the prognostic contributions of 15 covariates included in cause-specific Cox models of all-cause death and nonfatal MI during 5-year follow-up in CATHGEN in accordance with one embodiment of the disclosure.

FIG. 11 is a table showing the prognostic contributions of the six MVX variables to cause-specific Cox models of all-cause death and nonfatal MI during 5-year follow-up in CATHGEN in accordance with one embodiment of the disclosure.

FIG. 12 is a table showing the prognostic contributions of IVX and MMX to cause-specific Cox models of all-cause death and nonfatal MI during 5-year follow-up in CATHGEN in accordance with one embodiment of the disclosure.

FIG. 13 is a table showing the prognostic contributions of MVX to cause-specific Cox models of all-cause death and nonfatal MI during 5-year follow-up in CATHGEN in accordance with one embodiment of the disclosure.

FIG. 14 is a table showing the associations of different measures of HDL with 5-year mortality in CATHGEN in accordance with one embodiment of the disclosure. Only small HDL particles (under ˜8.8 nm diameter) were protective and effect-modified by GlycA.

FIG. 15 is a table showing the baseline characteristics of men and women participants in CATHGEN in accordance with one embodiment of the disclosure.

FIG. 16 is a table showing the prognostic contributions of IVX and MMX to Cox models for occurrence of all-cause death within 1 year, 3 years, 5 years, and between 6 to 10 years of follow-up in CATHGEN in accordance with one embodiment of the disclosure.

FIG. 17 is a table showing the prognostic contributions of MVX to Cox models for occurrence of all-cause death within 1 year, 3 years, 5 years, and between 6 to 10 years of follow-up in CATHGEN in accordance with one embodiment of the disclosure.

FIG. 18 is a table showing the prognostic contributions of IVX and MMX to cause-specific Cox models of different causes of death during 5-year follow-up in CATHGEN in accordance with one embodiment of the disclosure.

FIG. 19 is a table showing the prognostic contributions of MVX to cause-specific Cox models of different causes of death during 5-year follow-up in CATHGEN in accordance with one embodiment of the disclosure.

FIG. 20 is a table showing the associations of MVX scores with 5-year mortality in CATHGEN patient subgroups in accordance with one embodiment of the disclosure.

FIG. 21 is a set of plots showing the relative contribution and sign of association of each variable to the prediction of all-cause mortality in the Intermountain Heart cohort, calculated as percent of the total chi-square of the Cox regression model in accordance with one embodiment of the disclosure.

FIG. 22 is a set of graphs showing the cumulative mortality incidence by baseline MVX quintile in the development CATHGEN cohort and replication Intermountain Heart cohort in accordance with one embodiment of the disclosure.

The foregoing and other objects and aspects of the present disclosure are explained in detail in the specification set forth below.

### DETAILED DESCRIPTION

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present disclosure. The sequence of operations (or steps) is not limited to the order presented in the claims or figures unless specifically indicated otherwise.

The term “programmatically” means carried out using computer program and/or software, processor or ASIC directed operations. The term “electronic” and derivatives thereof refer to automated or semi-automated operations carried out using devices with electrical circuits and/or modules rather than via mental steps and typically refers to operations that are carried out programmatically. The terms “automated” and “automatic” means that the operations can be carried out with minimal or no manual labor or input. The term “semi-automated” refers to allowing operators some input or activation, but the calculations and signal acquisition as well as the calculation of the concentrations of the ionized constituent(s) are done electronically, typically programmatically, without requiring manual input.

The term “about” refers to +/−10% (mean or average) of a specified value or number.

The term “patient” or “subject” is used broadly and refers to an individual who provides a biological specimen sample, or a biosample, for testing or analysis.

The term “biosample” refers to in vitro blood, plasma, serum, CSF, saliva, lavage, sputum, or tissue samples of humans or animals. Embodiments of the disclosure may be particularly suitable for evaluating human blood plasma or serum biosamples, particularly for GlycA (which is not found in urine, for example). The blood plasma or serum samples may be fasting or non-fasting. The human biosample, particularly, may be most suitably obtained from the patient by way of phlebotomy.

The term “GlycA” refers to a biomarker that is derived from a measure of composite NMR signal from carbohydrate portions of acute phase reactant glycoproteins containing N-acetylglucosamine and/or N-acetylgalactosamine moieties, more particularly from the protons of the 2-NAcGlc and 2-NAcGal methyl groups. A GlycA signal is centered at about 2.00 ppm in a plasma NMR spectrum at about 47 degrees C. (+/−0.5 degrees C.). The peak location is independent of spectrometer field but may vary depending on analysis temperature of the biosample and is not found in urine biosamples. Thus, the GlycA peak region may vary if the temperature of the test sample varies. The GlycA NMR signal may include a subset of NMR signals at the defined peak region so as to include only clinically relevant signal contributions and may exclude a protein contribution to the signal in this region as will be discussed further below. See U.S. Pat. Nos. 9,361,429, 9,470,771, and 9,792,410, the contents of which are hereby incorporated by reference as if recited in full herein.

As used herein, the chemical shift locations (ppm) refer to NMR spectra referenced internally or externally. In an embodiment, the location may be referenced internally to CaEDTA signal at 2.519 ppm. Thus, the noted peak locations discussed and/or claimed herein may vary depending on how the chemical shift is generated or referenced as is well known to those of skill in the art. Thus, to be clear, certain of the described and/or claimed peak locations have equivalent different peak locations in other corresponding chemical shifts as is well known to those of skill in the art.

The terms “population norm” and “standard” refer to values defined by a large study or studies of higher-risk patients such as those enrolled in the Catheterization Genetics (CATHGEN) cardiac catheterization and Intermountain Heart Collaborative study biorepository, or other study having a large enough sample to be representative of the general population or targeted patient population. The instant disclosure is not limited to the population values in CATHGEN or Intermountain Heart Collaborative study as the presently defined normal or low-risk and high-risk population values as levels may change over time. Thus, a reference range associated with values from a defined population in risk segments (e.g., quartiles or quintiles) can be provided and used to assess elevated or reduced levels and/or risk of having a clinical disease state.

The term “clinical disease state” is used broadly and includes an at-risk medical condition that may indicate medical intervention, therapy, therapy adjustment or exclusion of a certain therapy (e.g., pharmaceutical drug) and/or monitoring is appropriate. Identification of a likelihood of a clinical disease can allow a clinician to treat, delay or inhibit onset of the condition accordingly.

As used herein, the term “NMR spectral analysis” means using proton (¹H) nuclear magnetic resonance spectroscopy techniques to obtain data that can measure the respective parameters present in the biosample, e.g., blood plasma or blood serum. “Measuring” and derivatives thereof refers to determining a level or concentration and/or for certain lipoprotein subclasses, measuring the average particle size thereof. The term “NMR derived” means that the associated measurement is calculated using NMR signal/spectra from one or more scans of an in vitro biosample in an NMR spectrometer.

The term “downfield” refers to a region/location on the NMR spectrum that pertains to the left of a certain peak/location/point (higher ppm scale relative to a reference). Conversely, the term “upfield” refers to a region/location on the NMR spectrum that pertains to the right of a certain peak/location/point.

The terms “mathematical model” and “model” are used interchangeably and when used with “MVX”, “metabolic vulnerability index”, or “risk”, refer to a statistical model of risk used to evaluate a subject's risk of premature mortality in the future, typically within 1-12 years. The risk model can be or include any suitable model including, but not limited to, one or more of a logistic regression model, a Cox proportional hazards regression model, a mixed model, or a hierarchical linear model. The risk models can provide a measure of risk based on the probability of premature mortality within a defined time frame, typically within 1-12 years. The risk models may be particularly suitable for providing risk stratification for patients having “intermediate risk” associated with a slight to moderate chance of having a clinical event based on traditional risk factors. The MVX risk model can stratify a relative risk of premature mortality as measured by standard χ2 and/or p values (the latter with a sufficiently representative study population).

The term “interaction parameter” refers to at least two different defined parameters combined as a (multiplied) product and/or ratio. Examples of interaction parameters include, but are not limited to, (S-HDLP)(GlycA) and (protein)(citrate).

The term “multimarker” refers to a multi-component biomarker.

The term “lipoprotein component” refers to a component in the mathematical risk model associated with lipoprotein particles including size and/or concentration of one or more subclasses (subtypes) of lipoproteins. Lipoprotein components can include any of the lipoprotein particle subclasses, concentrations, sizes, ratios and/or mathematical products (multiplied) of lipoprotein parameters and/or lipoprotein subclass measurements of defined lipoprotein parameters or combined with other parameters such as GlycA.

The term “HDLP” refers to a high-density lipoprotein particle number measurement (e.g., HDLP number) that sums the particle concentrations of defined HDL subclasses. Total HDLP can be generated using a total high density lipoprotein particle measurement that sums the concentration (μmol/L) of all the HDL subclasses (which may be grouped based on size into different size categories such as large, medium and small) in the size range between about 7 nm (on average) to about 14 nm (on average), typically between 7.4-13.5 nm. In some embodiments, HDL can be identified as a number of discrete size components, e.g., 7 subpopulations (H1-H7) of different sizes of HDLP ranging from a smallest HDLP size associated with H1 to a largest HDLP size associated with H7. In some embodiments, the defined subclass of HDL particles comprises small HDL particles (S-HDLP). In some embodiments, the S-HDLP can include HDL particle subclasses with diameters between about 7.3 nm (average) to about 9.0 nm (average).

The term MICS (malnutrition-inflammation complex syndrome) and MMIS (metabolic malnutrition-inflammation syndrome) can be used interchangeably and synonymously and used to describe the “reverse epidemiology” whereby increases in conventional cardiovascular risk factors such as body mass index (BMI), serum cholesterol, and blood pressure can be associated with decreased, rather than increased, cardiovascular and all-cause mortality.

The terms “sex” and “gender” are used interchangeably and synonymously herein. Thus, in many embodiments, examples, and figures, the terms “sex-specific” and “gender-specific” are used similarly and are used to describe individuals from each of the one or more cohorts. Additionally, and more specifically, as used herein and for the purposes of the study and the calculations, “sex” and “gender” have been limited to one of two options, male and female.

Abbreviated words can be defined as below unless otherwise specified throughout: ASCVD means atherosclerotic cardiovascular disease, CAD means coronary artery disease, CHF means chronic heart failure, CKD means chronic kidney disease, IVX means inflammation vulnerability index, MICS means malnutrition-inflammation complex syndrome, MMIS means metabolic-malnutrition inflammation syndrome, NMR means nuclear magnetic resonance, S-HDLP means small high-density lipoprotein particles, MMX means metabolic malnutrition index, MVX means metabolic vulnerability index.

### Lipoproteins

Lipoproteins can include a wide variety of particles found in plasma, serum, whole blood, and lymph, comprising various types and quantities of triglycerides, cholesterol, phospholipids, sphingolipids, and proteins. These various particles permit the solubilization of otherwise hydrophobic lipid molecules in blood and serve a variety of functions related to lipolysis, lipogenesis, and lipid transport between the gut, liver, muscle tissue and adipose tissue. In blood and/or plasma, lipoproteins can be classified in many ways, generally based on physical properties such as density or electrophoretic mobility or measures of apolipoprotein content, such as apoB or apoA-1, the main proteins in LDL and HDL, respectively.

Classification based on nuclear magnetic resonance-determined particle size can distinguish distinct lipoprotein particles based on size or size ranges. For example, the NMR measurements can identify at least 15 distinct lipoprotein particle subtypes, including at least 7 subtypes of high density lipoproteins (HDL), at least 3 subtypes of low density lipoproteins (LDL), and at least 5 subtypes of very low density lipoproteins (VLDL), which can also be designated TRL (triglyceride rich lipoprotein).

Current analysis methodology can allow NMR measurements that can provide concentrations of subpopulations of VLDL, LDL, and HDL to produce measurements of groups of small and large subpopulations of respective groups. For example, to optimize risk association with premature all-cause mortality, different size groupings of HDL subpopulations can be used as will be discussed further below.

The NMR derived estimated lipoprotein sizes noted herein typically refer to average measurements, but other size demarcations may be used.

In preferred embodiments, the MVX risk assessment model parameters can include NMR derived measurements of deconvolved signal associated with a common NMR spectrum of lipoproteins, and particularly HDLs, using defined deconvolution models that characterize deconvolution components for protein and lipoproteins, including HDL, LDL, VLDL/TRL. This type of analysis can provide for a rapid acquisition time of under 2 minutes, typically between about 20s-90s, and corresponding rapid programmatic calculations to generate measurements of the model components, then programmatic calculation of one or more MVX risk scores using one or more defined risk models.

Further, it is also noted that while NMR measurements of the lipoprotein particles can be contemplated as being particularly suitable for the analyses described herein, it is contemplated that other technologies may be used to measure these parameters now or in the future and embodiments of the disclosure are not limited to this measurement methodology. It is also contemplated that different protocols using NMR may be used (e.g., including different deconvolving protocols) in lieu of the deconvolving protocol described herein. See, e.g., Kaess et al., The lipoprotein subfraction profile: heritability and identification of quantitative trait loci, J Lipid Res. Vol. 49 pp. 715-723 (2008); and Suna et al., 1H NMR metabolomics of plasma lipoprotein subclasses: elucidation of metabolic clustering by self-organizing maps, NMR Biomed. 2007; 20: 658-672. Flotation and ultracentrifugation employing a density-based separation technique for evaluating lipoprotein particles and ion mobility analysis are alternative technologies for measuring lipoprotein subclass particle concentrations.

Lipoprotein subclass groupings can, for example, be summed to determine HDL or LDL particle numbers according to some particular embodiments of the present disclosure. It is noted that the “small, large, and medium” size ranges noted can vary or be redefined to widen or narrow the upper or lower end values thereof or even to exclude certain ranges within the noted ranges. The particle sizes noted above typically refer to average measurements, but other demarcations may be used.

Embodiments of the disclosure classify lipoprotein particles into subclasses grouped by size ranges based on functional/metabolic relatedness as assessed by their correlations with lipid and metabolic variables. Thus, as noted above, the evaluations can measure over 15 discrete subpopulations (sizes) of lipoprotein particles. These discrete sub-populations can be grouped into defined subclasses for VLDL/TRL and HDL and LDL. Intermediate-density lipoprotein (IDL) can be combined with VLDL/TRL or LDL or as a separate category in the size range between large LDL and small VLDL. For example, HDL subclass particles typically range (on average) from between about 7 nm to about 15 nm, more typically about 7.3 nm to about 14 nm (e.g., 7. 4 nm-13.5 nm). Total HDL concentration is the sum of the particle concentrations of the respective subpopulations of its HDL subclasses. The different subpopulations of HDLP can be identified by a number from 1-7, with “H1” representing the smallest-size HDL subpopulation and “H7” being the largest-size HDL subpopulation. In some embodiments, the defined subclass of HDL particles comprises small HDL particles (S-HDLP). In some embodiments, the S-HDLP can include HDL particle subclasses with diameters between about 7.3 nm (average) to about 9.0 nm (average).

### BCAAs

In some embodiments, the MVX model includes a measurement of at least one BCAA, as described in U.S. Pat. No. 9,361,429 and U.S. Pat. App. 20150149095, incorporated by reference herein. The MVX models may include one or more BCAAs including one or more of isoleucine, leucine, and valine (as discussed herein). In some embodiments, one or more of the three BCAAs (valine, leucine, and isoleucine) may be quantified by NMR.

### Ketone Bodies

In some embodiments, the MVX model may include a measurement of at least one ketone body ((3-hydroxybutyrate, acetoacetate, acetone), which can be obtained via NMR analysis of the biosample NMR spectrum. NMR quantification of each of the 3 ketone bodies is based on their NMR signal amplitudes as derived from separate deconvolution models specific to the 3 spectral regions in which the ketone body NMR signals appear. Deconvolution analysis, rather than simple integration of the ketone body signals, may be utilized because of extensive overlap with signals from numerous lipoprotein subspecies and identified and unidentified small molecule metabolites. The derived amplitudes of the β-hydroxybutyrate, acetoacetate, and acetone signals can be converted to μmol/L units of concentration using conversion factors determined by spiking serum with stock ketone body solutions of known concentration.

In one embodiment, a P-hydroxybutyrate methyl signal doublet appearing at approximately 1.16 and 1.15 ppm can be quantified using a lineshape deconvolution model that encompasses a spectral region from 1.07 to 1.33 ppm. This region can include the overlapped interfering NMR signals from lipid fatty acid methylene protons of numerous TRL, LDL, and HDL lipoprotein subspecies, serum protein signals, a triplet signal from ethanol (at 1.13, 1.15, and 1.17 ppm), a doublet signal from lactate (at 1.29 and 1.31 ppm), and a doublet signal from an unidentified metabolite (at 1.10 and 1.11 ppm) that appears only rarely in human serum specimens. In one embodiment, the deconvolution model can include a library of 83 spectral components to accurately account for the amplitudes of the NMR signals from β-hydroxybutyrate and the various interfering substances in serum.

In one embodiment, an acetoacetate methyl signal singlet appearing at approximately 2.24 ppm can be quantified using a lineshape deconvolution model that can encompass a spectral region from 2.22 to 2.39 ppm. This region can include the overlapped interfering NMR signals from lipid fatty acid methylene protons of numerous TRL, LDL, and HDL lipoprotein subspecies, serum protein signals, one or more octet signals from f3-hydroxybutyrate (2.25 to 2.39 ppm), and signals from 3 unidentified metabolites appearing at 2.22, 2.30, and 2.35-2.41 ppm. In one embodiment, the deconvolution model can include a library of 82 spectral components to accurately account for the amplitudes of the NMR signals from acetoacetate and the various interfering substances in serum.

In one embodiment, an acetone methyl signal singlet appearing at 2.19 ppm can be quantified using a line shape deconvolution model that encompasses the spectral region from 2.14 to 2.22 ppm. This region can include the overlapped interfering NMR signals from lipid fatty acid methylene protons of numerous TRL, LDL, and HDL lipoprotein subspecies, serum protein signals, and a singlet signal from an unidentified metabolite at 2.22 ppm. In one embodiment, the deconvolution model can include a library of 70 spectral components to accurately account for the amplitudes of the NMR signals from acetone and the various interfering substances in serum.

### GlycA

A defined mathematical lineshape deconvolution model can be used to measure the GlycA as described in U.S. Pat. No. 9,470,771 incorporated by reference in its entirety herein. The GlycA measurement can be a unitless parameter as assessed by NMR by calculating an area under a peak region at a defined peak location in NMR spectra. In any event, measures of GlycA with respect to a known population can be used to define the level or risk for certain subgroups, e.g., those having values within the upper half of a defined range, including values in the third and fourth quartiles, or the upper 3-5 quintiles and the like.

### Citrate

In some embodiments, the MVX model may include a measurement of citrate, which can be obtained via NMR analysis of the biosample NMR spectrum. NMR quantification of citrate can be based on the NMR signal amplitudes of three of the four members of a methylene proton quartet appearing at approximately 2.64, 2.60, and 2.48 ppm as can be derived from a deconvolution model that assumes a linear baseline and variable offset. The fourth member of the citrate signal quartet can appear at approximately 2.52 ppm and can overlap with a singlet signal from CaEDTA that serves as an internal chemical shift reference. The derived amplitudes of the citrate signals can be converted to μmol/L units of concentration using conversion factors determined by spiking serum with stock citrate solutions of known concentration.

### Serum Protein

In some embodiments, the MVX model can include a measurement of serum protein, which can be obtained via NMR analysis of the biosample NMR spectrum. Alternatively, serum protein or serum albumin measurements, where used, can be obtained in conventional ways. NMR quantification of serum protein can be based on the amplitude of the broad NMR signal from non-lipoprotein proteins derived from a lineshape deconvolution model that encompasses a spectral region from 0.71 to 1.03 ppm. This region includes the overlapped interfering NMR signals from lipid fatty acid methyl protons of numerous TRL, LDL, and HDL lipoprotein subspecies as well as those from the branched-chain amino acids valine, leucine, and isoleucine. In one embodiment, the deconvolution model can include a library of 66 spectral components to accurately account for the amplitudes of the NMR signals from serum protein and the various interfering substances in serum. The derived amplitude of the serum protein signal can be reported in arbitrary units of signal amplitude or converted to molar units of concentration using conversion factors determined by spiking serum with stock serum albumin solutions of known concentration.

### Methods

In an embodiment, a method comprises evaluating an NMR spectrum of at least one biomarker comprising: a high-density lipoprotein (HDL), GlycA, a Branched Chain Amino Acid, a Ketone, a Citrate, or a Protein from a patient sample. In a further embodiment, a plurality of the foregoing biomarkers is evaluated. In another embodiment, a method comprises evaluating an NMR spectrum of at least one biomarker comprising a high-density lipoprotein (HDL), GlycA, a Branched Chain Amino Acid, or a Citrate from a patient sample. In an additional embodiment, the result(s) of the evaluation of an NMR of one, or a plurality of the foregoing biomarkers, is/are used to create a score illustrative of the patient's risk of mortality and/or relative risk of mortality as compared to other patients.

In an embodiment of the present invention, a high-density lipoprotein (HDL) biomarker can include a wide variety of particles found in plasma, serum, whole blood, and lymph, comprising various types and quantities of triglycerides, cholesterol, phospholipids, sphingolipids, and proteins. The HDL biomarker may comprise an HDL subclass including, but not limited to, large HDL particles (L-HDLP) comprising a diameter of 9.5-12.0 nm; small HDL particles (S-HDLP) comprising a diameter of 7.4-8.7 nm; and the like.

In an embodiment, amino acids can be utilized to aid in generating the MVX index, where a Branched Chain Amino Acid biomarker may comprise, amino acids including, but not limited to, valine, leucine, and/or isoleucine or any combination thereof.

In an embodiment, a glycoprotein biomarker may comprise one or more acute-phase glycoproteins that may comprise at least GlycA.

In an embodiment, an organic biomarker may comprise at least a citrate molecule.

In an embodiment, a lipoprotein particle may comprise a high-density lipoprotein (HDL) biomarker and more specifically a small high-density lipoprotein particle.

In an embodiment, a plasma protein biomarker may comprise a plasma protein subclass including, but not limited to, albumin.

In an embodiment, a ketone body biomarker may comprise a subset of ketone bodies, including but not limited to acetone, acetoacetate, or beta-hydroxybutyrate.

As noted, the results of the evaluation of a biomarker or biomarkers in an embodiment of the invention may be utilized to create a score illustrative of a patient's risk and/or relative risk of mortality. U.S. Pat. No. 11,156,621, the disclosure of which is hereby incorporated by reference, includes a discussion of a “Metabolic Vulnerability index” (MVX). The MVX score may be calculated using one or more NMR derived measurements from an embodiment of the invention. The one or more NMR derived measurements may include a proton NMR spectrum resulting from at least one BCAA biomarker, at least one HDL biomarker, at least one glycoprotein biomarker, a citrate biomarker, a plasma protein biomarker, and a ketone body biomarker, or a combination thereof.

In embodiments of the disclosure, at least one or more biomarkers individually or in combination, when analyzed in NMR spectroscopy, provide data to illustrate a patient's risk and/or relative risk of mortality. When calculating MVX to demonstrate risk and/or relative risk of mortality in a patient, biomarkers and subclasses of biomarkers may be used. In yet other embodiments, when calculating MVX to demonstrate risk and/or relative risk of mortality, other biomarkers not included herein may be utilized and analyzed using NMR spectroscopy to generate an MVX score. Enhanced multi-marker performance may be achieved by inclusion of additional biomarkers.

Embodiments of the disclosure include methods of determining the levels of markers associated with a person's risk of premature death. The methods may include obtaining a sample from the person and measuring GlycA, at least one high density lipoprotein particle (HDLP) subclass, at least one branched chain amino acid (BCAA), at least one ketone body, at least one serum protein, and citrate. The methods may further include obtaining a sample from the person and measuring GlycA, at least one high density lipoprotein particle (HDLP) subclass, at least one branched chain amino acid (BCAA), and citrate. In an embodiment the high-density lipoprotein particle is a small HDL particle (S-HDLP).

In some embodiments, these measurements are used to generate a MVX score. In some embodiments, the MVX score is determined by using the following model: MVX=A+β1*lnGlycA+β2*lnS-HDLP+β4*lnBCAA+β5*lnKetoneBody. In some embodiments, the MVX value is determined by using the following model: MVX=A+β1*lnGlycA+(32*lnS-HDLP+β3*(lnGlycA*lnS-HDLP)+β4*lnBCAA+β5*lnKetoneBody.

It is noted that throughout this disclosure, the empirical values for A and β₁-βₙ may vary depending upon the model used. For example, β1 in the first above formula for MVX (i.e., the formula that does not include the term β3*(lnGlycA*lnS-HDLP)), will generally be a different value than β1 in the second above formula (i.e., the equation that does include that product term), respectively.

In some embodiments, the methods include measuring at least one of citrate in addition to GlycA, at least one HDLP subclass, and at least one BCAA. In some embodiments, the methods include simultaneously measuring citrate, GlycA, at least one HDLP subclass, and at least one BCAA. In some other embodiments, the methods include measuring at least one of citrate and protein in addition to GlycA, at least one HDLP subclass, at least one BCAA, and at least one ketone body. In some embodiments, the measuring that includes at least one of citrate (Citrate) and serum protein (Protein) is performed on a subject deemed to be at high risk for death. In some embodiments, the MVX value is determined using the following model: MVX=A+β1*lnGlycA+β2*lnS-HDLP+β3*(lnGlycA*lnS-HDLP)+(34*1nBCAA+β5*lnKetoneBody+β6*lnCitrate+β7*lnProtein+β8*(lnCitrate*lnProtein).

In some embodiments, the MVX value is defined as comprising an inflammation vulnerability index (IVX) value and a metabolic malnutrition index (MMX) value. The values MVX, IVX, and MMX can all be sex-specific. For example, MVX_{F}, a value specific to females, may be defined as comprising the female-specific value MMX_{F} and IVX_{F}. Alternatively, MVX_{M}, can be a value specific to males, and may be defined as comprising the male-specific value MMX_{M} and IVX_{M}. Generally, the MVX value can be defined as comprising an inflammation vulnerability index (IVX) value and a metabolic malnutrition index (MMX) value regardless of sex.

In some embodiments, the measurements of at least GlycA and the at least one HDLP subclass are used to generate an inflammation vulnerability index (IVX) value. In some embodiments, the IVX value is determined using the following model: IVX=β1*lnGlycA+β2*lnS-HDLP+β3*(lnGlycA*lnS-HDLP). In other embodiments, the IVX_{F} value can be determined using the following model: 9+(GlycA*−0.000187)+(S-HDLP*−0.3585)+((GlycA*S-HDLP)*0.000348). Within the IVX_{F}, a resulting score can vary but can include a range of 3.0 to 9.0 inclusive, corresponding to a score of 1 to 100, respectively. Additionally, the IVX_{M} can be determined using the following model: 9+(GlycA*−0.00437)+(S-HDLP*−0.52307)+((GlycA*S-HDLP)*0.000817). Within the IVX_{M}, a resulting score can vary but can generally include a range of 1.4 to 7.6 inclusive, corresponding to a score of 1 to 100, respectively.

In some embodiments, the measurements of at least one BCAA and at least one ketone body are used to generate a metabolic malnutrition index (MMX) value. In some embodiments, the MMX value is determined using the following model: MMX=β4*lnBCAA+β5*lnKetoneBody. This model may be denoted MMX1 and, as discussed in detail herein, be the calculation used for populations/subjects that are believed to be low risk.

In other embodiments, the measurements of at least one BCAA, at least one ketone body, citrate, and protein are used to generate an alternate MMX value. For example, in some embodiments, the MMX value may be determined using the following model: MMX=β4*lnBCAA+β5*lnKetoneBody+β6*lnCitrate+β7*lnProtein. Or, the MMX value may be determined using the following model: MMX=β4*lnBCAA+β5*lnKetoneBody+β6*lnCitrate+β7*lnProtein+(38*(lnCitrate*lnProtein). In such embodiments, MMX may be described as follows: MMX=(39*MMX1+β10*MMX2, where MMX1 is as described above, and MMX2=β6*lnCitrate+β7*lnProtein+(38*(lnCitrate*lnProtein).

The measurements used to generate the MMX value including measurements of citrate and protein are generally performed in a subject and/or population deemed to be at high risk for CVD-related death (e.g., subjects who have had a cardiovascular event or symptoms suggestive of underlying CVD).

Yet in other embodiments of the present disclosure, the MMX value can be determined using at least one BCAA and citrate. The MMX value can be sex-specific. Thus, the MMX_{F} value can be found using the following model:
((4+(Leu*−0.03142)+(Leu²*0.0000893))*0.353)+((7+(Val*−0.03362)+(Val²*0.0000689))*0.684)+(Ileu*0.00332)+((1+(Citr*−0.0072)+(Citr²*0.0000573))*0.7135).

Within the MMX_{F}, a resulting score can vary but can generally include a range of ln (0.8) to ln (1.8) inclusive, corresponding to a score of 1 to 100 respectively. Additionally, the MMXₘ can be determined using the following model:
((4+(Leu*−0.01594)+(Leu²*0.0000291))*1.076)+((7+(Val*−0.0239)+(Val²*0.00005))*0.414)+(Ileu*0.01265)+((1+(Citr*0.00906)+(Citr²*−0.0000126))*0.5881).

Within the MMX_{M}, a resulting score can vary but can generally include a range of ln (1.59) to ln (2.1) inclusive, corresponding to a score of 1 to 100 respectively.

Thus, in some embodiments, the metabolic vulnerability index may be described as: MVX=βi*IVX+βm*MMX. For application to normal (i.e., low) risk patient populations (e.g., people who have not had a known CV event) the metabolic vulnerability index may be described as MVX1=βi*IVX+βm*MMX1 (wherein βi and βm may have unique values depending upon the model used). Conversely, for application to high-risk patient populations (e.g., people who have had a known CV event) the metabolic vulnerability index may be described as MVX=βi*IVX+βm*MMX wherein βi and βm may have unique values depending upon the model used and MMX=β9*MMX1+β10*MMX2. In some cases, βm is the same for both the low-risk and the high-risk models.

In some embodiments, the metabolic vulnerability index for females may be described as: MVX_{F}=(IVX_{F}*2.27278)+(lnMMX_{F}*12.13511)+(IVX_{F}*lnMMX_{F})*−1.09312 where the resulting score can vary but can generally include 17 to 25 inclusive, corresponding to a score of 1 to 100, respectively. Additionally, the metabolic vulnerability index for males maybe described as:
MVX_{M}=(IVX_{M}*3.54601)+(lnMMX_{M}*14.41428)+(IVX_{F}*lnMMX_{M})*−1.43438 where the resulting score can vary but can generally include 27 to 34.2 inclusive, corresponding to a score of 1 to 100, respectively.

For example, in some cases the MVX score may be monitored in a high-risk subject or population as a way to monitor the subject's overall health and risk for a fatal CV event or death from other causes. Or, the MVX score may be monitored in a clinical trial for a new pharmaceutical that is being performed in a high-risk population as a way to monitor the efficacy of the test drug. For low-risk subjects, the clinician may choose to monitor the MVX value as a means to assess general health and wellness. The MVX value may, in certain embodiments, be used as a guide to lifestyle changes that promote cardiac health.

### Systems

Still other embodiments are directed to a system. Some embodiments of the disclosure comprise a system capable of performing each method described herein. In some embodiments, the system may comprise an NMR spectrometer configured to acquire an NMR spectrum and/or spectra comprising at least one signal for GlycA, at least one signal for at least one small high density lipoprotein particle (S-HDLP) subclass, at least one signal for at least one branched chain amino acid (BCAA), and at least one signal for at least one citrate; and a processor to determine a metabolic vulnerability index (MVX) value based on the measured at least one signal for the GlycA, the at least one small high density lipoprotein particle (S-HDLP) subclass, the at least one branched chain amino acid (BCAA), and the at least one citrate wherein the processor comprises or communicates with a memory.

In some embodiments, the system includes an NMR spectrometer for acquiring at least one NMR spectrum of an in vitro biosample and at least one processor in communication with the NMR spectrometer. The at least one processor may be configured to determine for a respective biosample using the at least one NMR spectrum a metabolic vulnerability index score based on at least one defined mathematical model of risk of premature mortality that may consider at least one HDL subclass component measurement, at least one branched chain amino acid measurement, a GlycA measurement, a citrate measurement, and optionally a ketone body and/or a protein measurement obtained from at least one in vitro biosample of the subject. In an embodiment, the HDLP subclass is small HDLP (S-HDLP). In some embodiments, the processor may be configured to calculate an MVX score based on the measurements of GlycA, at least one ketone body, at least one branched chain amino acid, and the at least one of the HDLP subclass using the following formula: MVX=A+β1*lnGlycA+β2*lnS-HDLP+β4*lnBCAA+β5*lnKetoneBody. In some embodiments, the processor may be configured to calculate the MVX score using the following model: MVX=A+β1*lnGlycA+β2*lnS-HDLP+β3*(lnGlycA*lnS-HDLP)+β4*lnBCAA+β5*lnKetoneBody. In some embodiments, the processor may be configured to calculate an MVX score using the following model: MVX=A+β1*lnGlycA+β2*lnS-HDLP+β3*(lnGlycA*lnS-HDLP)+β4*lnBCAA+β5*ln KetoneBody+β6*lnCitrate+β7*lnProtein+β8*(lnCitrate*lnProtein). In some embodiments, the processor may be configured to calculate a sex-specific MVX score from one of the following models:
MVX_{F}=(IVX_{F}*2.27278)+(lnMMX_{F}*12.13511)+(IVX_{F}*lnMMX_{F})*−1.09312 where the resulting score can vary but can generally include 17 to 25 inclusive, corresponding to a score of 1 to 100, respectively. Or for males: MVX_{M}=(IVX_{M}*3.54601)+(lnMMX_{M}*14.41428)+(IVX_{F}*lnMMX_{M})*−1.43438 where the resulting score can vary but can generally include 27 to 34.2 inclusive, corresponding to a score of 1 to 100, respectively.

In some embodiments, the processor may be configured to calculate an MVX score comprising an inflammation vulnerability index (IVX) value and a metabolic malnutrition index (MMX) value. Thus, in some embodiments, the processor may be configured to calculate an MVX score based on the following model : MVX=(3i*IVX+(3m*MMX. In these embodiments, the processor may be configured to calculate an IVX value using the following model: IVX=β1*lnGlycA+β2*lnS-HDLP+β3*(lnGlycA*lnS-HDLP). In some embodiments, the processor may be configured to calculate an MMX value using the following model: MMX=β4*lnBCAA+β5*lnKetoneBody; this model may be denoted MMX1. In some embodiments, the processor may be configured to calculate an MMX using the following model: MMX=β4*lnBCAA+β5*lnKetoneBody+β6*lnCitrate+β7*lnProtein. In another embodiment, the processor may be configured to calculate the MMX using the following model: MMX=β4*lnBCAA+β5*lnKetoneBody+β6*lnCitrate+β7*lnProtein+β8*(lnCitrate*lnProtein). In such embodiments, MMX may be described as follows: MMX=β9*MMX1+β10*MMX2, where MMXI is as described above, and MMX2=β6*lnCitrate+β7*lnProtein+(38*(lnCitrate*lnProtein).

In some embodiments, the processer can be configured to calculate a sex-specific MMX value using the following models:
MMX_{F}=((4+(Leu*−0.03142)+(Leu²*0.0000893))*0.353)+((7+(Val*−0.03362)+(Val²*0.0000689))*0.684)+(Ileu*0.00332)+((1+(Citr*−0.0072)+(Citr²*0.0000573))*0.7135).

Within the MMX_{F}, a resulting score can vary but can generally include a range of ln (.8) to ln (1.8) inclusive, corresponding to a score of 1 to 100 respectively.
MMX_{M}=((4+(Leu*−0.01594)+(Leu²*0.0000291))*1.076)+((7+(Val*−0.0239)+(Val²*0.00005))*0.414)+(Ileu*0.01265)+((1+(Citr*0.00906)+(Citr²*−0.0000126))*0.5881).

Within the MMX_{M}, a resulting score can vary but can generally include a range of ln (1.59) to ln (2.1) inclusive, corresponding to a score of 1 to 100 respectively.

Still other embodiments are directed to NMR systems. The systems include a NMR spectrometer; a flow probe in communication with the spectrometer; and at least one processor in communication with the spectrometer. The at least one processor may be configured to obtain (i) at least one NMR signal of a defined GlycA fitting region of NMR spectra associated with GlycA of a blood plasma or serum specimen in the flow probe; (ii) at least one NMR signal of a defined citrate fitting region of NMR spectra associated with the specimen in the flow probe; (iii) at least one NMR signal of a defined BCAA fitting region of NMR spectra associated with the specimen in the flow probe; and (iv) at least one NMR signal of HDLP subclass parameters. The processor may be further configured to calculate measurements of the GlycA, the citrate, the at least one branched chain amino acid, and the HDLP subclass parameters. The system may be further configured to calculate a MVX score using a defined mathematical model of risk of all cause-mortality that uses the calculated measurements of GlycA, citrate, at least one branched chain amino acid, at least one of the HDLP subclass parameters, and optionally serum protein (Protein) and/or a ketone body. In an embodiment, the HDLP subclass is small HDLP (S-HDLP). In some embodiments, the system may be further configured to calculate an MVX score based on the measurements of GlycA, citrate, at least one branched chain amino acid, and the at least one of the HDLP subclass. In some embodiments, the system may be further configured to calculate an MVX score based on the measurements of GlycA, at least one ketone body, at least one branched chain amino acid, and the at least one of the HDLP subclass using the following formula: MVX=A+β1*lnGlycA+β2*lnS-HDLP+β4*lnBCAA+β5*lnKetoneBody. In some embodiments, the system may be further configured to calculate the MVX score using the following model: MVX=A+β1* lnGlycA+β2*lnS-HDLP+β3*(lnGlycA*lnS-HDLP)+β4*lnBCAA+β5*lnKetoneBody. In some embodiments, the system may be further configured to calculate an MVX score using the following model: MVX=A+β1* lnGlycA+β2*lnS-HDLP+β3*(lnGlycA*lnS-HDLP)+β4*lnBCAA+β5*ln KetoneBody+β6*lnCitrate+β7*lnProtein+(38*(lnCitrate*lnProtein). In some embodiments, the system may be further configured to calculate an MVX score comprising an inflammation vulnerability index (IVX) value and a metabolic malnutrition index (MMX) value. In some embodiments, the system may be configured to calculate a sex-specific MVX score from one of the following models using sex-specific IVX and MMX:
MVX_{F}=(IVX_{F}*2.27278)+(lnMMX_{F}*12.13511)+(IVX_{F}*lnMMX_{F})*31 1.09312 where the resulting score can vary but can generally include 17 to 25 inclusive, corresponding to a score of 1 to 100, respectively. Or for males:
MVX_{M}=(IVX_{M}*3.54601)+(lnMMX_{M}*14.41428)+(IVX_{F}*lnMMX_{M})*−1.43438 where the resulting score can vary but can generally include 27 to 34.2 inclusive, corresponding to a score of 1 to 100, respectively.

Thus, in some embodiments, the system may be further configured to calculate an MVX score based on the following model : MVX=βi*IVX+βm*MMX. In these embodiments, the system may be further configured to calculate an IVX value using the following model: IVX=β1*lnGlycA+β2*lnS-HDLP+β3*(lnGlycA*lnS-HDLP). In some embodiments, the system may be further configured to calculate an MMX value using the following model: MMX=β4*lnBCAA+β5*lnKetoneBody; this model may be denoted MMX1. In some embodiments, the system may be further configured to calculate an MMX using the following model: MMX=β4*lnBCAA+β5*lnKetoneBody+β6*lnCitrate+β7*lnProtein. In another embodiment, the system may be further configured to calculate the MMX using the following model: MMX=β4*lnBCAA+β5*lnKetoneBody+β6lnCitrate+β7*lnProtein+(38*(lnCitrate*lnProtein). In such embodiments, MMX may be described as follows: MMX=β9*MMX1+β10 *MMX2, where MMX1 is as described above, and MMX2=β6*lnCitrate+β7*lnProtein+(38*(lnCitrate*lnProtein).

### Additional Methods

Additional aspects of the present disclosure are directed to methods of monitoring a patient to evaluate a therapy or determine whether the patient is at-risk of premature mortality. The methods may include: programmatically providing at least one defined MVX mathematical model as disclosed herein that includes a plurality of components including NMR derived measurements of at least one selected HDLP subclass, at least one of a branched chain amino acid, a citrate, and GlycA and optionally at least one of protein or at least one ketone body. The method may further comprise programmatically deconvolving a spectrum comprising the NMR derived measurements. The method may also comprise programmatically calculating a MVX score of the respective patients using the at least one defined model and corresponding patient sample measurements; and evaluating at least one of (i) whether the MVX score is above a defined level of a population norm associated with increased risk of all-cause mortality; and/or (ii) whether the metabolic vulnerability index is increasing or decreasing over time in response to a therapy. In some embodiments, the MVX score may be calculated based on the measurements of GlycA, at least one ketone body, at least one branched chain amino acid, and the at least one of the HDLP subclass using the following formula: MVX=A+β1*lnGlycA+β2*lnS-HDLP+β4*lnBCAA+β5*lnKetoneBody. In some embodiments, the MVX score may be calculated using the following model: MVX=A+β1*lnGlycA+β2*lnS-HDLP+β3*(lnGlycA*lnS-HDLP)+β4*lnBCAA+β5*lnKetoneBody. In some embodiments, the MVX score may be calculated using the following model: MVX=A+β1*lnGlycA+β2*lnS-HDLP+β3*(lnGlycA*lnS-HDLP)+β4*lnBCAA+β5*ln KetoneBody+β6*lnCitrate+β7*lnProtein+β8*(lnCitrate*lnProtein). In some embodiments, the MVX score calculation may comprise an inflammation vulnerability index (IVX) value and a metabolic malnutrition index (MMX) value. Thus, in some embodiments, the calculation of an MVX score may be based on the following model: MVX=βi*IVX+βm*MMX. In these embodiments, the calculation of an IVX value may use the following model: IVX=β1*lnGlycA+β2*lnS-HDLP+β3*(lnGlycA*lnS-HDLP). In some embodiments, the calculation of an MMX value may use the following model: MMX =β4*lnBCAA+β5*lnKetoneBody; this model may be denoted MMX1. In some embodiments, the calculation of an MMX score may the following model: MMX=β4*lnBCAA+β5*lnKetoneBody+β6*lnCitrate+β7*lnProtein. In another embodiment, the calculation of the MMX score may use the following model: MMX=β4*lnBCAA+β5*lnKetoneBody+β6*lnCitrate+β7*lnProtein+(38*(lnCitrate*lnProtein). In such embodiments, MMX may be described as follows: MMX=(39*MMX1+β10*MMX2, where MMX1 is as described above, and MMX2=β6*lnCitrate+β7*lnProtein+(38*(lnCitrate*lnProtein).

Herein are disclosed methods and systems to determine a subject's metabolic vulnerability index (MVX). The methods comprise utilizing a multi-variate model of defined biomarkers to predict a patient's chance of premature all-cause mortality in Malnutrition-inflammation complex syndrome (MICS), also referred to as metabolic malnutrition-inflammation syndrome (MMIS).

One embodiment of the disclosure utilizes a method of determining the levels of marker associated with a subject's relative risk of premature death comprising obtaining a sample from a subject; measuring one or more markers using NMR spectroscopy wherein the markers can include GlycA, at least one high density lipoprotein particle (HDLP) subclass, at least one branched chain amino acid (BCAA), citrate, and optionally at least one ketone body (KetoneBody), and at least one subset of serum protein; determining a metabolic vulnerability index based on the NMR spectra; repeat sample processing over time; evaluate at least whether the MVX has increased or decreased over time.

In an embodiment, the HDLP is a small HDLP (S-HDLP).

In some embodiments, the MVX score may be sex-specific. In some embodiments, the MVX score can be calculated using IVX and MMX. In some embodiments, the MMX score can be calculated using at least one BCAA and citrate. In some embodiments, the IVX score can be calculated using a GlycA measurement and an HDLP measurement.

In some embodiments, IVX is sex-specific. In the same embodiments, MMX is also sex-specific. In some embodiments, HDLP is a small HDLP (S-HDLP) however, in other embodiments, HDLP can be a medium or a large HDLP (M-HDLP, L-HDLP).

In some embodiments, the MVX score may be calculated based on the measurements of GlycA, at least one ketone body, at least one branched chain amino acid, and the at least one of the HDLP subclass using the following formula: MVX=A+β1*lnGlycA+β2*lnS-HDLP+β4*lnBCAA+β5*lnKetoneBody.

In some embodiments, the MVX score may be calculated using the following model: MVX=A+β1*lnGlycA+β2*lnS-HDLP+β3*(lnGlycA*lnS-HDLP)+β4*lnBCAA+β5*lnKetoneBody. In these embodiments, the MVX value may be determined in a subject deemed to be at low risk for a cardiovascular event.

In some embodiments, the MVX value may include a measurement of serum protein (Protein) and/or citrate (Citrate).

In some embodiments, the MVX value may be determined using the following model: MVX=A+β1* lnGlycA+β2*lnS-HDLP+β3*(lnGlycA*lnS-HDLP)+β4*lnBCAA+β5*lnKetoneBody+β6*lnCitrate+β7*lnProtein+(38*(lnCitrate*lnProtein). In these embodiments, the MVX value may be determined in a subject deemed to be at high risk for a cardiovascular event.

In an alternative embodiment, the MVX value may comprise an inflammation vulnerability index (IVX) and/or a metabolic malnutrition index (MMX) as disclosed in more detail herein.

Thus, in some embodiments, the calculation of an MVX score may be based on the following model: MVX=(3i*IVX+(3m*MMX. In these embodiments, the calculation of an IVX value may use the following model: IVX=β1*lnGlycA+β2*lnS-HDLP+β3*(lnGlycA*lnS-HDLP).

In some embodiments, the calculation of an MMX value may use the following model: MMX=β4*lnBCAA+β5*lnKetoneBody; this model may be denoted MMX1 and be used for subjects deemed to be at low risk for a cardiovascular disease-related event.

In some embodiments, the calculation of an MMX score may the following model: MMX=β4*lnBCAA+β5*lnKetoneBody+β6*lnCitrate+β7*lnProtein.

In another embodiment, the calculation of the MMX score may use the following model: MMX=β4*lnBCAA+β5*lnKetoneBody+β6*lnCitrate+β7*lnProtein+β8*(lnCitrate*lnProtein).

In such embodiments, MMX may be described as follows: MMX=(39*MMX1+β10*MMX2, where MMX1 is as described above, and MMX2=β6*lnCitrate+β7*lnProtein+β8*(lnCitrate*lnProtein); in these embodiments, the MVX score may be determined for subjects deemed to be at high risk for a cardiovascular disease-related event.

In some embodiments, the BCAA may be at least one of leucine, isoleucine, or valine.

In some embodiments, the ketone bodies may be at least one of acetone, acetoacetate, or beta-hydroxybutyrate.

In some embodiments, measuring is performed by NMR. The metabolic vulnerability index can provide short (1 year) to long (12 year) term premature death risk assessments. A follow up with a subject may take place after 12 years, after 10 years, and/or every 6 years, every 5 years, every 4 years, every 3 years, every 2 years, or every 1 years. These risk assessments can generate MVX values decoupled from traditional risk factors.

In some embodiments, sex may be included as a factor in the MVX model.

In some embodiments, age may be included as a factor in the MVX model. In other embodiments, the MVX model can exclude either sex or age considerations so as to avoid generating false negatives or false positives based on data corruption of such ancillary data not directly tied to a biosample, for example.

In some embodiments, the MVX score is provided to the clinician based on data electronically correlated to the sample or based on clinician or intake lab input, e.g., fasting “F” or non-fasting “NF,” and statin “S” or non-statin “NS” characterizations of the patient which data can be provided on labels associated with the biosample to be electronically associated with the sample at the NMR analyzer. Alternatively, the patient characterization data can be held in a computer database (remote or via server or other defined pathway) and can include a patient identifier, sample type, test type, and the like entered into an electronic correlation file by a clinician or intake laboratory that can be accessed by or hosted by the intake laboratory that communicates with the NMR analyzer. The patient characterization data can allow the appropriate MVX model to be used for a particular patient.

It is contemplated that a metabolic vulnerability index can be used to monitor subjects in clinical trials and/or on drug therapies, to identify drug contradictions, and/or to monitor for changes in risk status (positive or negative) that may be associated with a particular drug, a patient's lifestyle and the like, which may be patient specific.

Referring some embodiments of the disclosure comprise an NMR system capable of performing each method described herein.

In some embodiments, the NMR system may comprise an NMR spectrometer; a flow probe in communication with the spectrometer; and a processor in communication with the spectrometer configured to obtain (i) at least one NMR signal of a defined GlycA fitting region of NMR spectra associated with GlycA of a blood plasma or serum specimen in the flow probe; (ii) at least one NMR signal of a defined citrate fitting region of NMR spectra associated with the specimen in the flow probe; (iii) at least one NMR signal of a defined BCAA fitting region of NMR spectra associated with the specimen in the flow probe; and, (iv) at least one NMR signal for at least one HDLP subclass; and optionally, at least one NMR signal for serum protein (Protein) and/or ketone body.

In some embodiments, the processor is further configured to calculate an MVX score based on measurements obtained by the spectrometer according to any of the embodiments of the invention disclosed herein.

Further embodiments of the disclosure will now be described by way of the following non-limiting Examples.

### EXAMPLES

The disclosure may be better understood by reference to the following non-limiting examples.

### Example 1

A composite biomarker score named the Metabolic Vulnerability Index (MVX) was derived from six metabolites measured simultaneously by a clinically-deployed nuclear magnetic resonance (NMR) blood test that likely reflect different etiologic aspects of metabolic malnutrition-inflammation syndrome(s). The MVX score provided strikingly strong stratification of all-cause mortality risk in two large, independent cohorts of cardiac catheterization patients not previously suspected of susceptibility to these dysmetabolic wasting syndromes. The contribution of MVX to multivariable prediction models of 5-year mortality dominated those of 15 risk factors including age. Risk associations were similarly strong in men and women, in younger and older individuals, those who were overweight and underweight, and in patients with and without comorbid conditions such as heart failure, renal dysfunction, diabetes, and hypertension. The uniformity of the MVX risk associations in higher- and lower-risk patient subgroups suggests the influence of metabolic malnutrition-inflammation syndromes on survival may be more universal than previously thought.

A factor that may affect risk of mortality may include malnutrition and inflammation among other potential factors. Further, there may be a correlation between malnutrition-inflammation and muscle decay that can impart further risk of mortality. Malnutrition-inflammation associated muscle decay can be described most often in patients with chronic kidney disease (CKD) or chronic heart failure (CHF). Malnutrition-inflammation associated muscle decay can also be noted in both healthy and frail individuals and in patients with malignancy and liver disease. Examples of the names given to the syndrome(s) can include protein-energy wasting (PEW) of which cachexia can be a severe form, malnutrition-inflammation atherosclerosis syndrome, and malnutrition-inflammation complex syndrome (MICS), the term favored that connotes a synergistic relationship between systemic inflammation and protein-energy malnutrition.

A subpopulation with a high prevalence of MICS can likely display a “risk factor paradox”, also called “reverse epidemiology”, whereby increases in conventional cardiovascular risk factors such as body mass index (BMI), serum cholesterol, and blood pressure can be associated with decreased, rather than increased, cardiovascular and all-cause mortality. Pathophysiologic mechanisms contributing to cardiovascular disease are not inoperative in the subpopulation, but a different superimposed etiology seemingly can predominate to drive a reversal of a plurality of usual risk factors associated with mortality. Thus, in patients with MICS, the usual risk factors of fatal versus nonfatal outcomes may be governed by different influences, and these may optimally respond to different therapeutic interventions.

A likely contributing impediment to greater awareness and understanding of MICS and its involvement in frailty syndromes can be a lack of simple quantitative and objective clinical assessment tools. The malnutrition component can be particularly challenging to assess, typically utilizing patient history, physical examination, and anthropomorphic evaluation. The main laboratory biomarkers of MICS, low serum albumin (reflecting both undernutrition and inflammation) and elevated C-reactive protein (CRP), can be nonspecific and therefore limited in their clinical applicability.

It is likely that all-cause mortality in a large, high-risk CATHGEN (CATHeterization GENetics) cardiac catheterization cohort may be independently associated with 2 novel biomarkers that might plausibly reflect inflammatory contributions to the MICS etiology. Both can be measured by nuclear magnetic resonance (NMR) spectroscopy as part of a clinical NMR LipoProfile® scan. The first can be GlycA, a composite NMR signal arising from the glycan residues of several acute-phase glycoproteins, thereby providing a sensitive and stable measure of systemic inflammation. The second can be the quantity of smaller-size high-density lipoprotein particles (S-HDLP) that can appear to mediate several protective functions carried out by (among others) bound anti-inflammatory and immune response proteins.

### Laboratory Methods

LipoProfile® analyses of fasting EDTA plasma samples are performed on the NMR Profiler platform at LipoScience (now Labcorp, Morrisville, NC) using an LP-4 algorithm. A “scan” (proton NMR spectrum) can produce one or more particle concentrations of several different-size subclasses of triglyceride-rich lipoproteins (TRL), low-density lipoproteins (LDL), high-density lipoproteins (HDL), mean TRL, LDL, and HDL particle sizes, plus derived lipids (triglycerides and total, LDL, and HDL cholesterol), the inflammation marker GlycA, 3 branched-chain amino acids (valine, leucine, isoleucine), citrate, plasma protein, ketone bodies, and several other small molecule metabolites. In some cases, seven HDL particle subspecies with an indicated estimated diameter (nm) were quantified: H7P (12 nm), H6P (10.8 nm), H5P (10.3 nm), H4P (9.5 nm), H3P (8.7 nm), H2P (7.8 nm), and H1P (7.4 nm). The identified subparticles were grouped for analysis purposes into “large” (L-HDLP=H4P+H5P+H6P+H7P) and “small” (S-HDLP=H1P+H2P+H3P) HDL subclasses. In the Intermountain Heart cohort, lipids are measured by standardized chemical analysis. Lipids and creatinine were measured by standardized chemical analysis and estimated glomerular filtration rate (eGFR) was calculated using a 2021 CKD-EPI equation. Chronic Kidney Disease (CKD) was defined as eGFR <60 ml/min/1.73 m².

### Study Population

For this study, consecutive patients undergoing cardiac catheterization at Duke University Medical Center for suspicion of ischemic heart disease enrolled in the CATHGEN biorepository between 2001 and 2011 with sufficient available frozen EDTA plasma were identified (n=6,969). The final study cohort (n=5,876) excluded those with missing angiographic (n=889), heart failure (n=98), creatinine (n=78), and BMI (n=28) information. Demographics, medical history, and angiographic data were obtained from the Duke Databank for Cardiovascular Disease. Follow-up included determination of mortality (confirmed through the National Death Index and the Social Security Death Index) and myocardial infarction (MI). Every individual was followed longitudinally with contact made at 6 months after the procedure and yearly thereafter. The CATHGEN biorepository is monitored and was approved by the Duke University Institutional Review Board on March 18, 2011. Before collection of blood samples, all study participants provided written informed consent. Incident events were defined as all-cause or cause-specific death or non-fatal MI at any time during the follow-up period and time to time event was defined as time enrolling cardiac catheterization to time of death at any point after enrollment. Median (IQR) follow-up time was 6.2 (4.4-8.9) years. Cardiovascular death was defined as death from 1 of the following causes: MI, heart failure, sudden death, postresuscitation, vascular cause, during or post cardiac surgery, or during cardiac catheterization. Noncardiovascular death was defined as death due to a noncardiac medical cause or a noncardiac cause related to a procedure. Unknown causes of death were defined as unobserved or unknown cause of death. Coronary artery disease (CAD) was defined as the presence of at least 1 epicardial coronary vessel with clinically significant stenosis (≥75%) at time of the index catheterization.

The second study population (n=2,998) was drawn from the Intermountain Heart Collaborative Study cardiac catheterization registry of patients that underwent coronary angiography (September 2000-September 2006) at the LDS Hospital (Salt Lake City, Utah) (2) Consecutive patients were included if they were ≥18 years of age, had at least 5 years follow-up, and had sufficient available frozen EDTA plasma and nonmissing clinical and laboratory variables. Patients provided informed consent prior to angiography and the study was approved by the Intermountain Urban Central Region Institutional Review Board on Mar. 23, 2012. Incident events were all-cause death determined by hospital records, Utah State Health Department records (death certificates), and the Social Security Administration death master file. Time to event was defined as the time from enrolling cardiac catheterization to time of death at any point after enrollment. Median (IQR) follow-up time was 8.2 (6.9-9.2) years.

### Statistical Analysis

Continuous variables may presented as mean ±SD or median and interquartile range (IQR) and dichotomous variables as percentages. A chi-square statistic and student's t-test are used to compare baseline characteristics among those who did and did not die during 5-year follow-up. Spearman correlation coefficients are used to assess correlations between selected variables. Associations of NMR-measured lipoprotein and metabolite variables with all-cause mortality (and additionally cause-specific mortality and nonfatal MI in CATHGEN) may be assessed using Cox proportional hazards models, adjusted for age, sex, race, smoking, diabetes, hypertension, BMI, total cholesterol, HDL cholesterol, triglycerides, eGFR, CAD, heart failure, prior MI, and family history of CAD. The assumption of proportional hazards is tested by including time-varying covariates in the models. Among several NMR measures found to have significant associations with all-cause mortality in CATHGEN when examined individually, including plasma protein (inverse) and ketone bodies, only 6 (S-HDLP, GlycA, citrate, valine, leucine, isoleucine) made significant independent contributions to a joint prediction model. These may be combined into sex-specific IVX, MMX, and MVX multimarker scores. Estimates of the relative importance of each predictive variable may be provided by its chi-square value as percent of the total chi-square of the model. Estimates of the relative importance may be very similar to those derived by comparing the discrimination c-index of the full model to those of models leaving out each variable. CATHGEN and Intermountain Heart statistical analyses may be performed by I.S. using SAS version 9.4 and by H.T.M. using SPSS version 22.0, respectively. All reported P values may be two-sided.

### Results

A study was conducted to evaluate relative risks of mortality in two large cohorts of patients. Samples from patients were collected from 5,876 of a CATHGEN cohort and 2,998 patients of an Intermountain Heart cohort over the course of 5 years. Samples optimally included blood samples that were drawn by way of phlebotomy. Samples were evaluated, using NMR spectroscopy, for the presence and amount of 8 health markers referred to as biomarkers. The 8 biomarkers included GlycA, at least one high density lipoprotein particle (HDLP) subclass, three branched chain amino acids (BCAA) including valine, leucine, and isoleucine, at least one ketone body, citrate, and at least one subset of serum protein. From the biomarkers, and at least 6 biomarkers including GlycA, Citrate, at least one high density lipoprotein particle (HDLP) subclass, and at least on BCCAA, utilizing the spectra from the NMR instrument previously obtained, three indices were calculated: metabolic malnutrition index (MMX), inflammation vulnerability index (IVX), and malnutrition vulnerability index (MVX) as a result of the prior two indices. The biomarkers were assessed over the course of a 5-year period and thereby the indices were also assessed. MVX, resulting from MMX and IVX, was tracked over the course of years in two large cohorts to assess risk of mortality in patients with malnutrition-inflammation complex syndrome (MICS). Interestingly, out of 8 biomarkers, only 6 of the analyzed biomarkers (S-HDLP, GlycA, citrate, valine, leucine, isoleucine) made significant independent contributions to a joint prediction model.

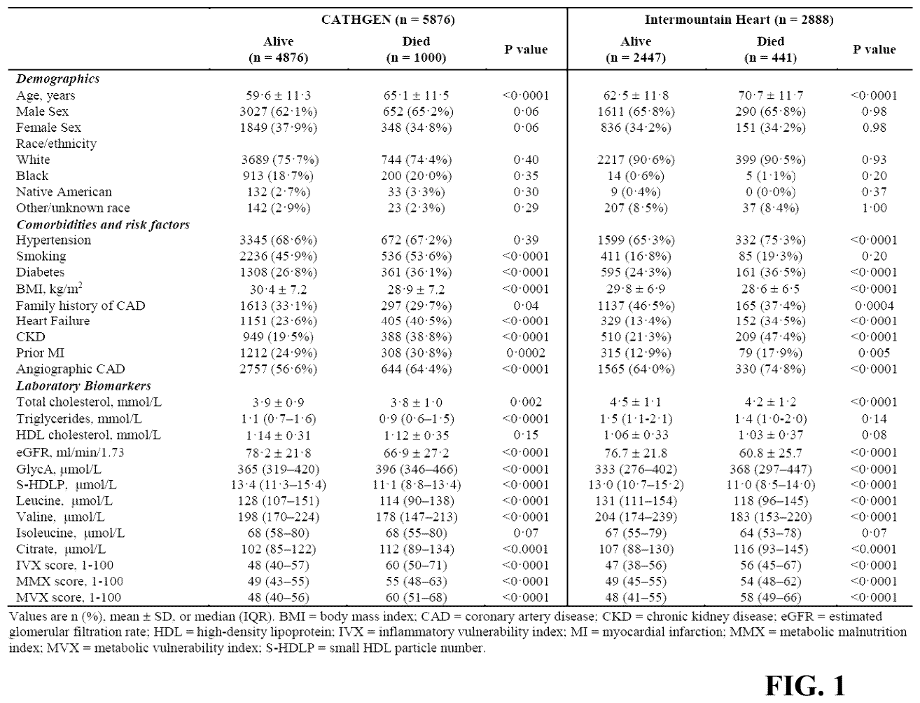

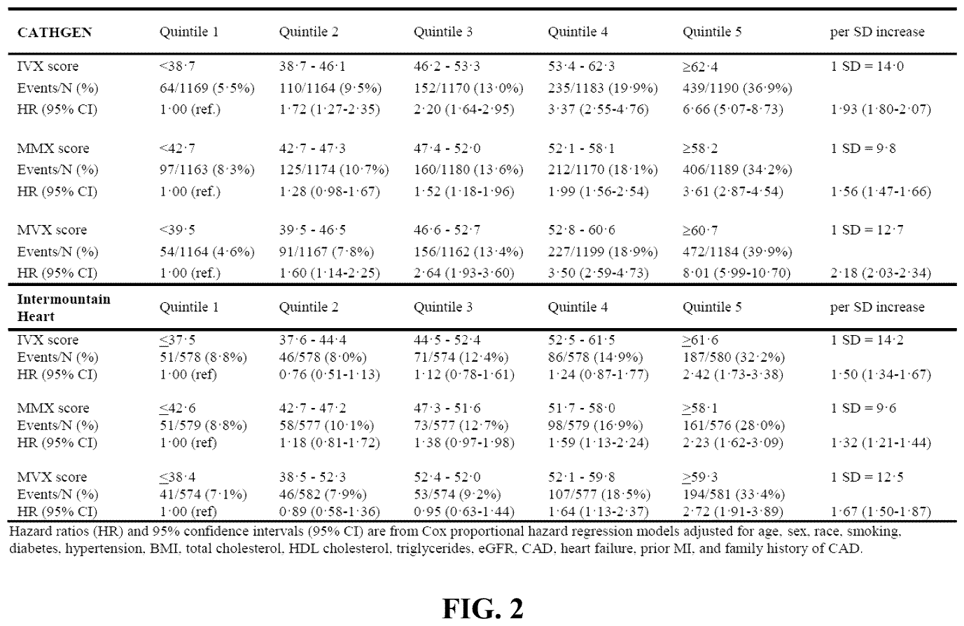

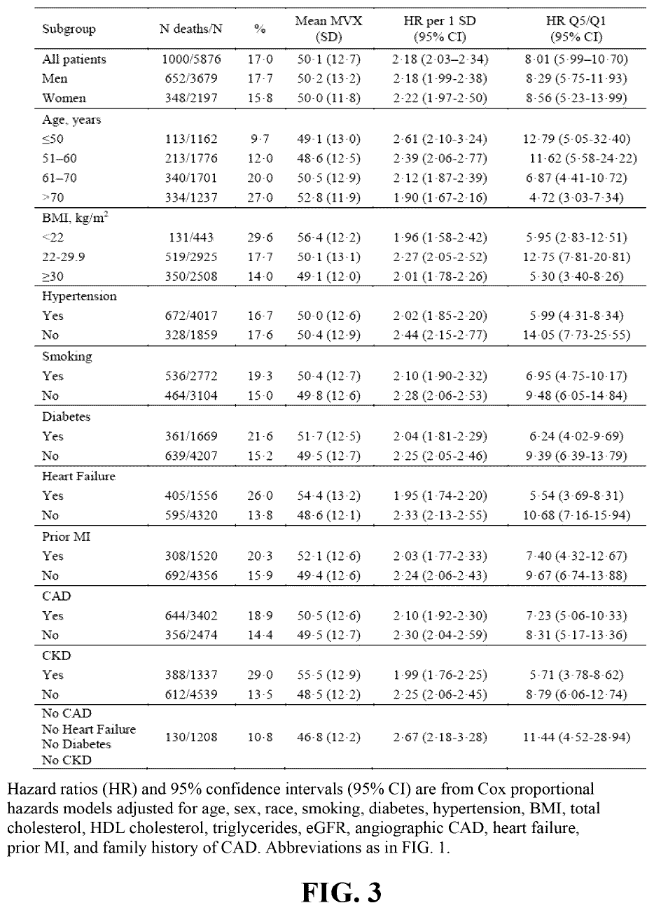

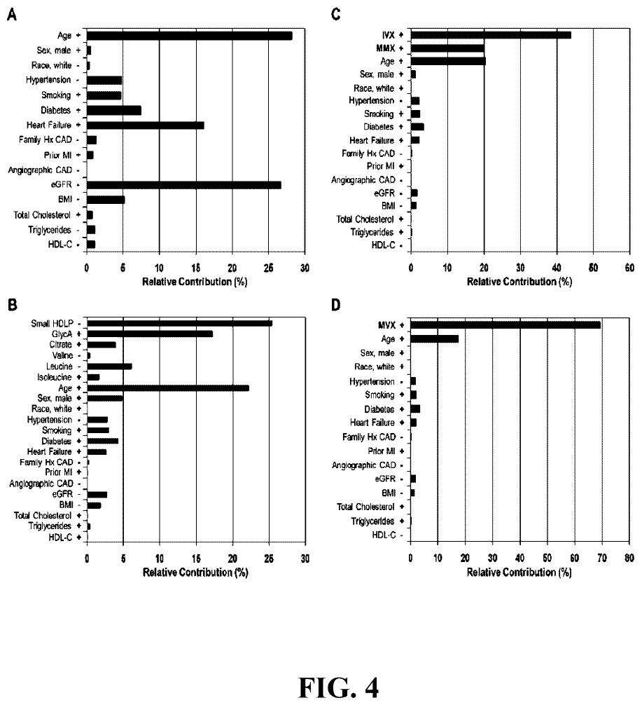

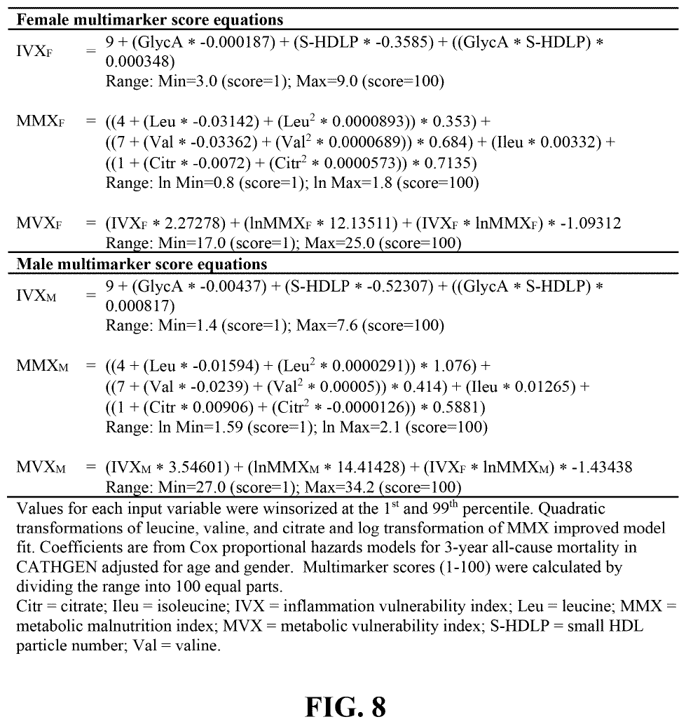

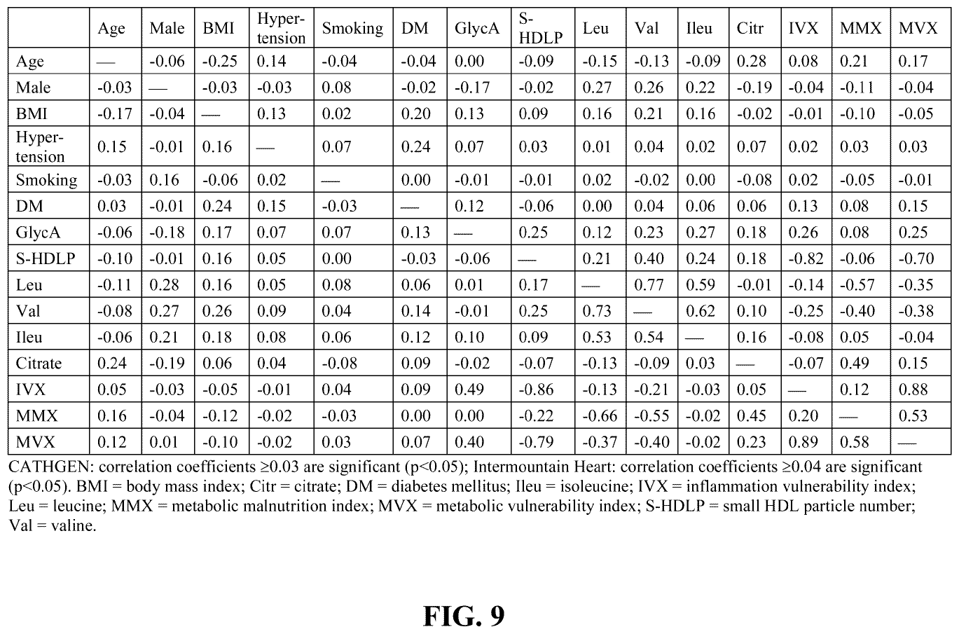

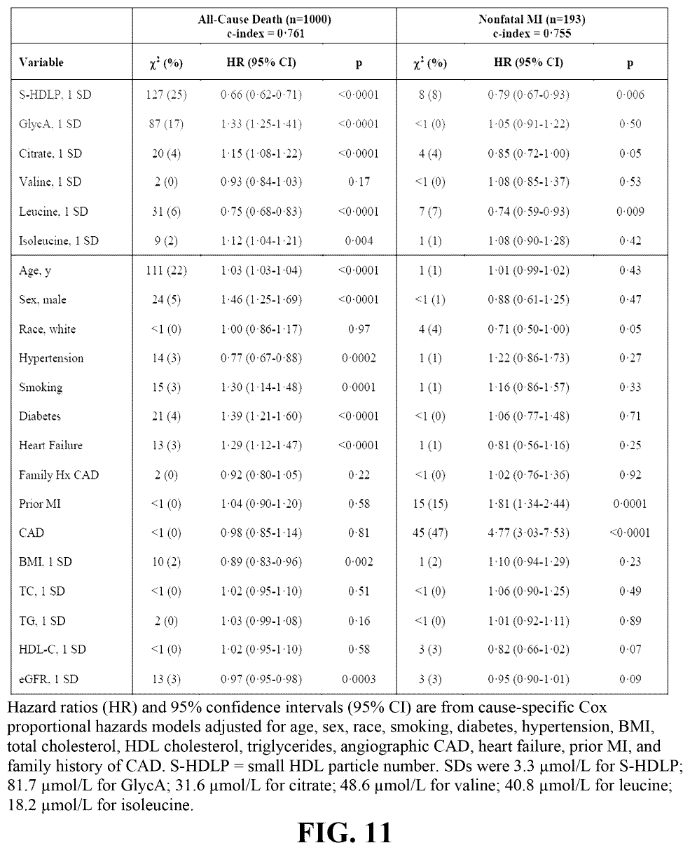

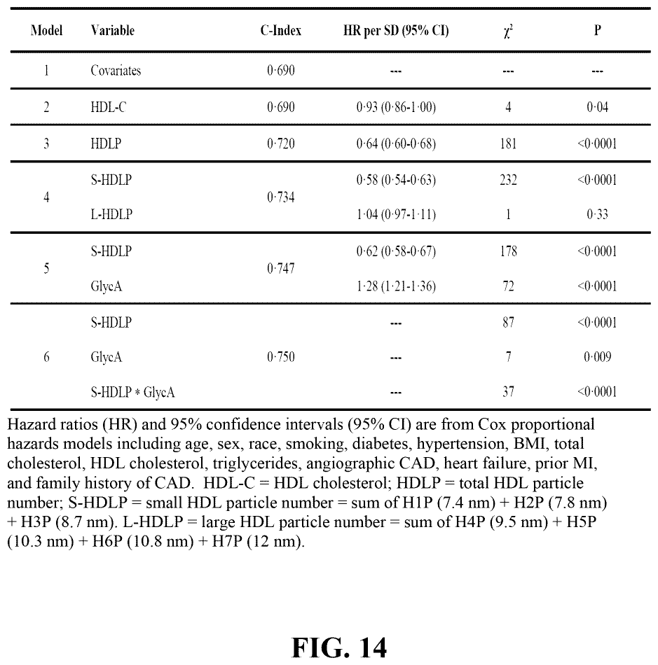

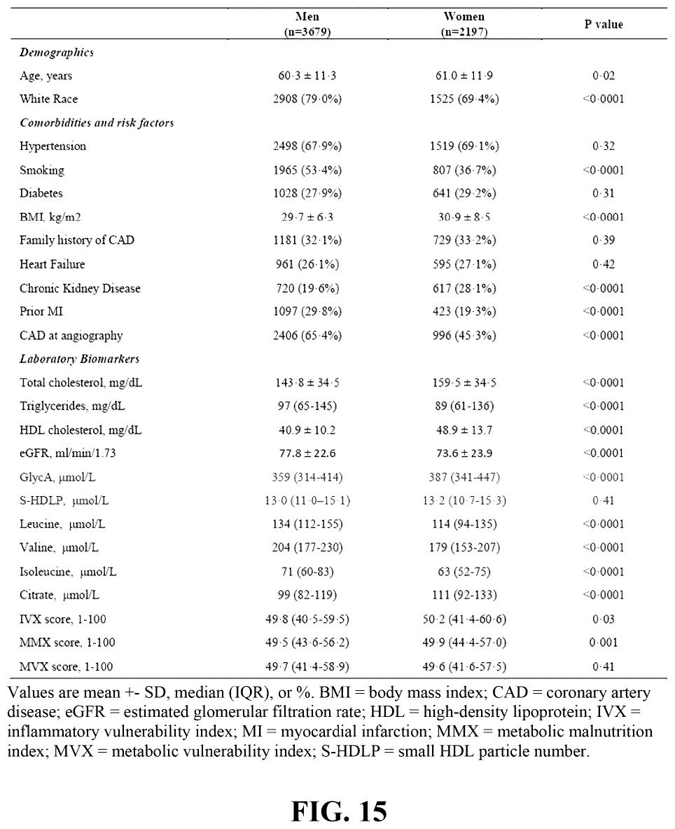

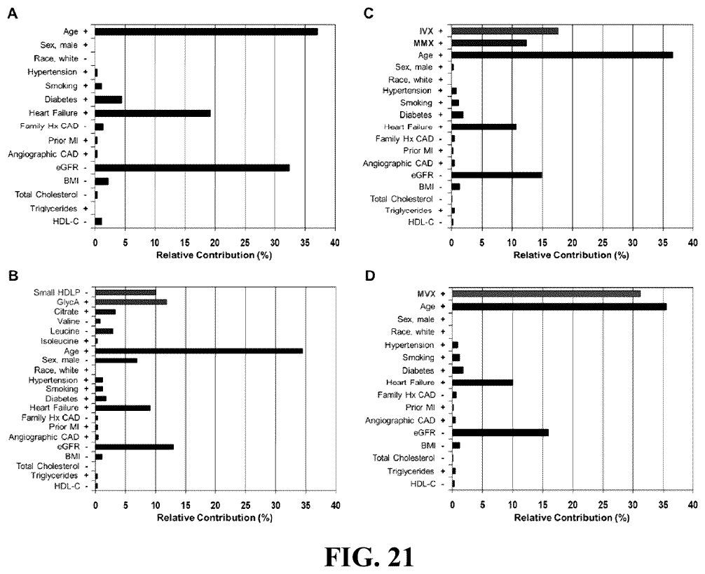

Of the 5,876 CATHGEN and 2,888 Intermountain Heart patients evaluated, 1,000 (17%) and 441 (15.3%), respectively, died within 5 years. Baseline characteristics of the participants stratified by 5-year survival status are presented in FIG. 1. In both cohorts there was a high prevalence of hypertension, diabetes, heart failure, and angiographically-defined CAD, plus signs of the “risk factor paradox” (lower cholesterol, triglycerides, and BMI in those who died). Among NMR-measured metabolites postulated to reflect MICS, levels of GlycA and citrate were higher and S-HDLP, valine, and leucine lower in patients that died. Correlations between these biomarkers and with selected risk factors were similar in both cohorts (FIG. 9),In CATHGEN, the 6 presumed MICS-related biomarkers made significant contributions to a multivariable prediction model of mortality, increasing the c-index from 0.690 to 0.761 (FIGS. 4, and 11). As estimated by the percent contribution made to the total chi-square of the model, the strongest base model predictors were age (28%), renal function assessed by eGFR (27%), and heart failure (16%). Addition of the MMIS biomarkers to the model reduced predictive contributions of heart failure and eGFR substantially (to 3% each), replaced in importance by 5-HDLP (25%) and GlycA (17%). The strong protective (inverse) association observed for small HDL particles applied only to particles under ˜8.8 nm diameter; concentrations of larger-size HDL particles and total HDL cholesterol (HDL-C) had negligible mortality associations (FIG. 14). In the Intermountain Heart replication cohort, mortality associations of the 6 MMIS variables were similar to those in CATHGEN, through major contributions of heart failure and eGFR were attenuated less (9% from 19% and to 13% from 32%, respectively) and GlycA (12%) and S-HDLP (12%) were relatively reduced in importance (FIG. 21). Given the complexity and multifactorial etiology of the metabolic dysfunctions underlying cachexia/sarcopenia/malnutrition and its associated mortality risk, utility is plausible for producing 2 mortality multimarker “subscores”: the Inflammation Vulnerability Index (IVX) combining GlycA and S-HDLP and the Metabolic Malnutrition Index (MMX) combining valine, leucine, isoleucine, and citrate (FIG. 8) These were calculated sex-specifically to make scores numerically similar in men and women, which they otherwise would not be owing to sex differences in metabolite levels unrelated to mortality risk. Specifically, citrate and GlycA levels are higher and BCAA levels lower in women (FIG. 15).

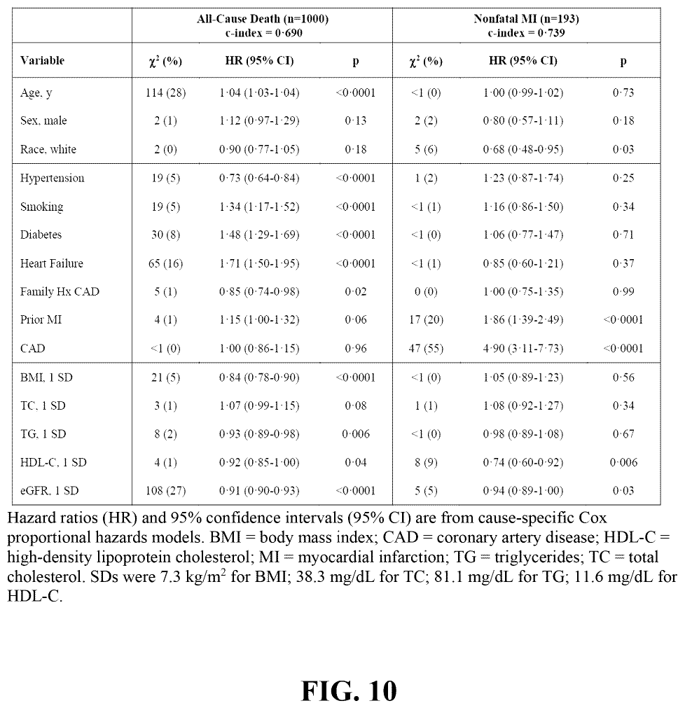

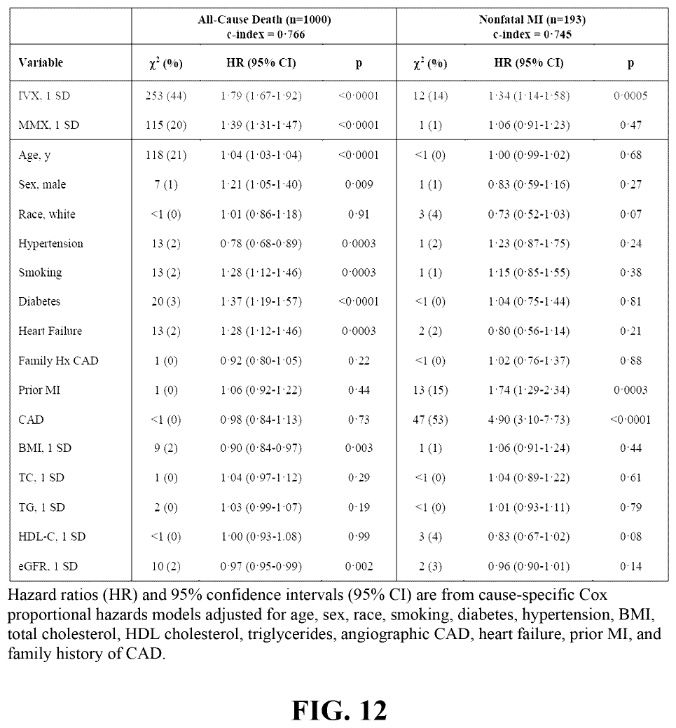

In CATHGEN, the predictive contribution of IVX (44%) was about twice that of MMX (20%) (FIGS. 4 and 12), whereas in Intermountain Heart IVX and MMX made comparable contributions to mortality risk of 18% and 12%, respectively (FIG. 21). In contrast to mortality prediction, the risk of nonfatal MI was much less influenced by the MMIS-related NMR biomarkers and much more dependent on comorbid conditions, with the combination of prevalent angiographic CAD and prior history of MI making dominant contributions (62-75%) in CATHGEN (FIGS. 4 and 10).

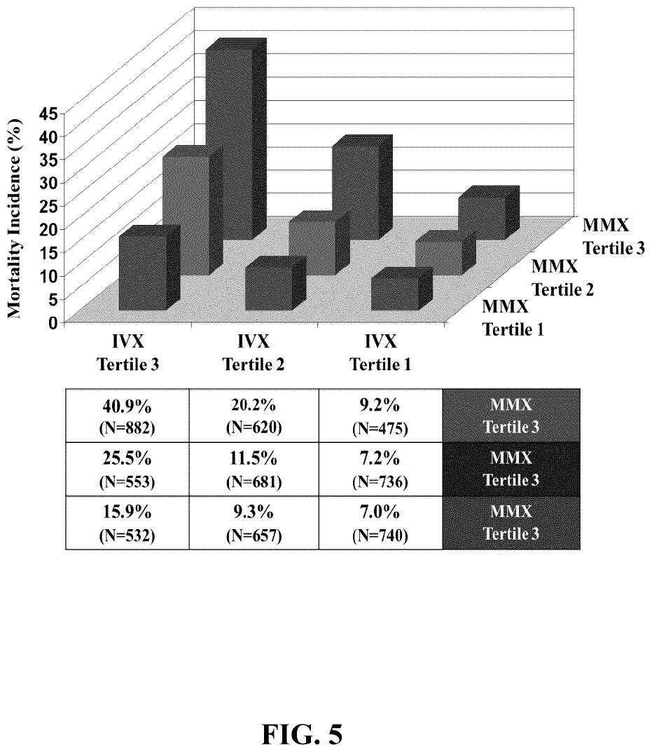

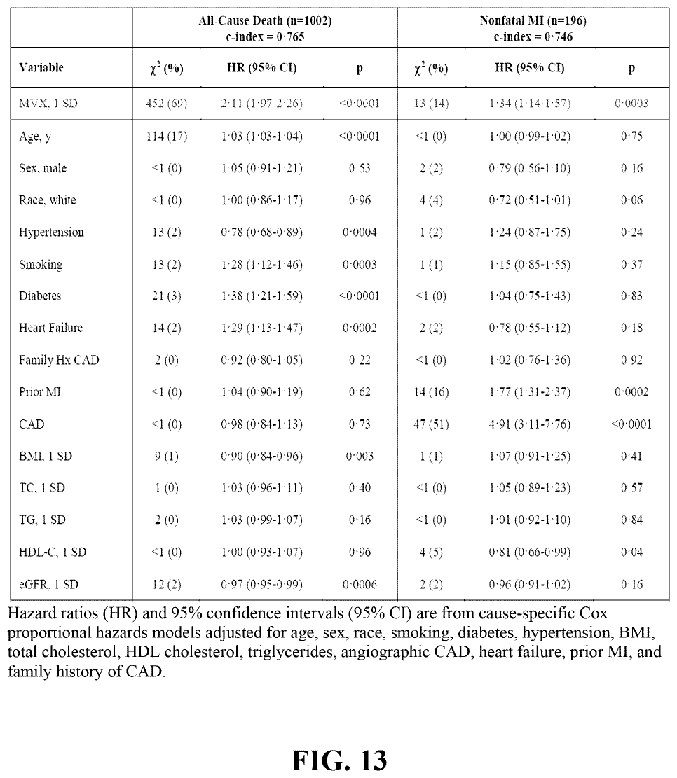

Combining IVX and MMX produces the overall mortality risk multimarker named MVX (Metabolic Vulnerability Index). The calculation of MVX includes a product term (IVX*MMX) accounting for observed interaction (synergy) between the inflammation and malnutrition parts of MICS (FIG. 8). A cross-classification graph of 5-year mortality by IVX and MMX tertile (FIG. 5) illustrates this interaction by showing that higher mortality risk in those with high MMX scores occurs only when IVX scores are also high. MVX in CATHGEN contributed far more to mortality prediction (69%) than any of the other 15 risk factors in the model, including age (17%) (FIGS. 4 and 13). In Intermountain Heart, MVX was also dominant, though less so, with its predictive contribution (31%) exceeding those of all variables except age (35%) (FIG. 21).

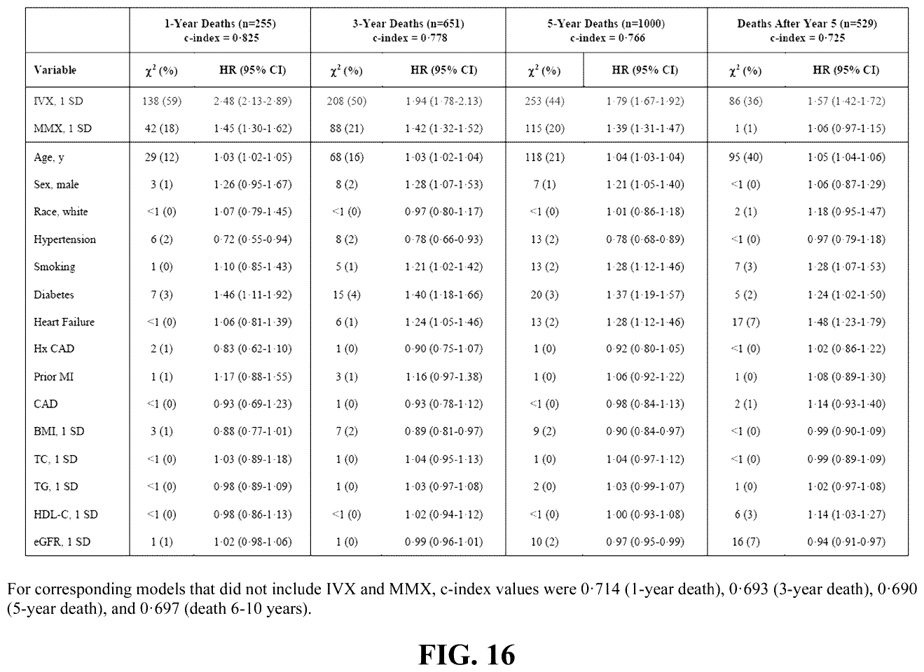

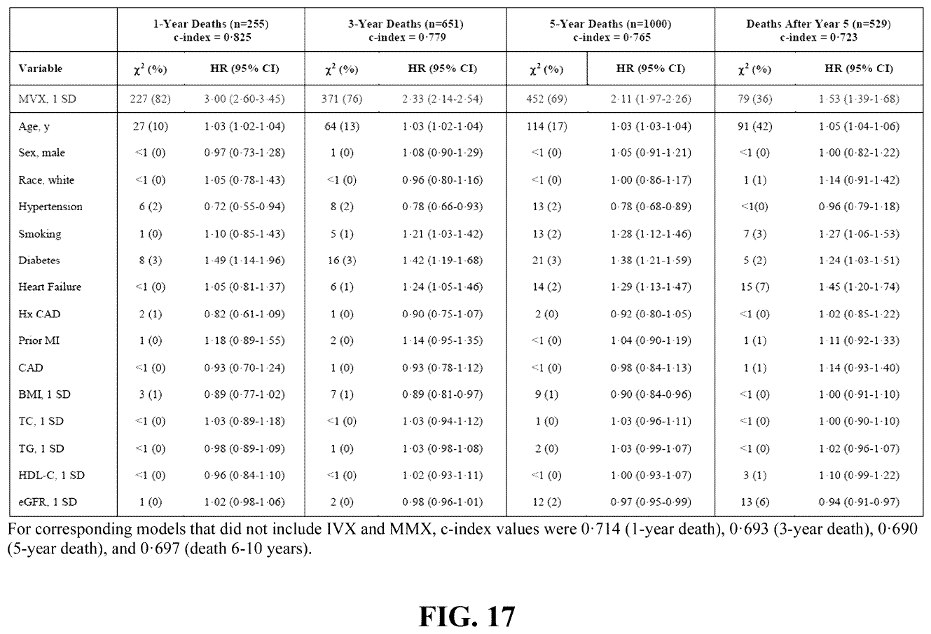

There was a deviation of the assumption of proportional hazards (p<0.0001) in models of 5-year mortality for IVX and MVX, but not MMX. A sensitivity analysis was therefore performed that compared models limited to mortality occurring within 1 year (n=255), 3 years (n=651), 5-years (n=1000) and between 6 to 10 years (n=529) of enrollment in CATHGEN (FIG. 16-17). IVX and MVX were associated most strongly with 1-year mortality (IVX HR: 2.48; 95% CI 2.13-2.89; MVX HR: 3.00; 95% CI 2.60-3.45), and remained dominant predictors of death occurring after 5 years (IVX HR: 1.57; 95% CI 1.42-1.72; MVX HR: 1.53; 95% CI 1.39-1.68). MMX, in contrast, displayed near constant mortality associations for the first 5 years of follow-up and weakened substantially longer-term. The contributions of MVX (82%) and age (10%) dominated prediction of 1-year mortality as well as death occurring after 5 years β6% and 42%, respectively).

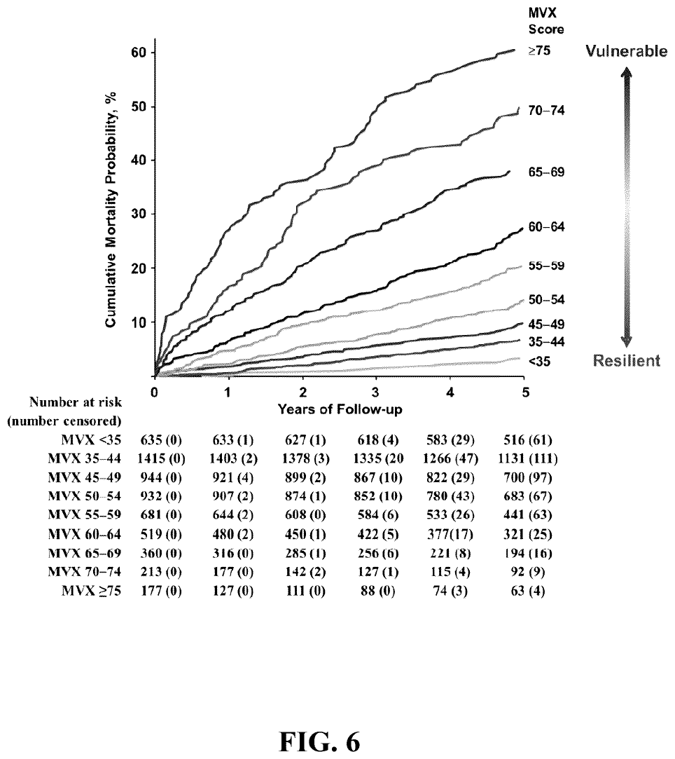

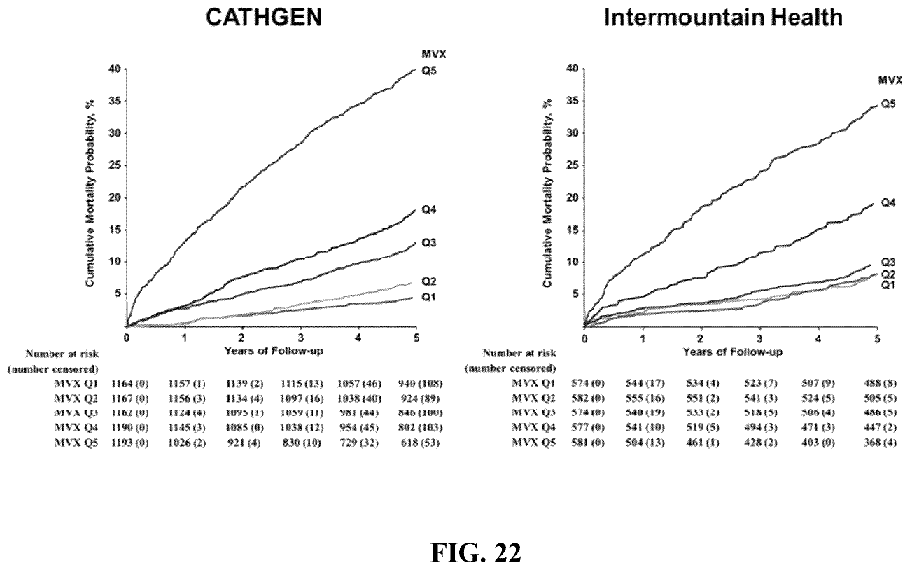

FIG. 2 shows (risk factor-adjusted) associations of IVX, MMX, and MVX with a year all-cause mortality in the CATHGEN and Intermountain Heart cohorts, examined in quintiles and per 1 SD. The significant associations of all 3 multimarkers in CATHGEN replicated in Intermountain Heart but were somewhat weaker (CATHGEN: MVX HR: 2.18; 95% CI 2.03-2.34 per 1 SD of 12.7; Intermountain: MVX HR: 1.67; 95% CI 1.50-1.87 per 1 SD of 12.5). A plot of cumulative mortality in subgroups of CATHGEN participants stratified by small differences in baseline MVX score shows a remarkably graded relationship, with clinically meaningful mortality differences seen not just for high values, but also lower values of MVX (FIG. 6). Risk stratification by MVX quintile in the Intermountain Heart cohort was comparable to that observed in CATHGEN (FIG. 22).

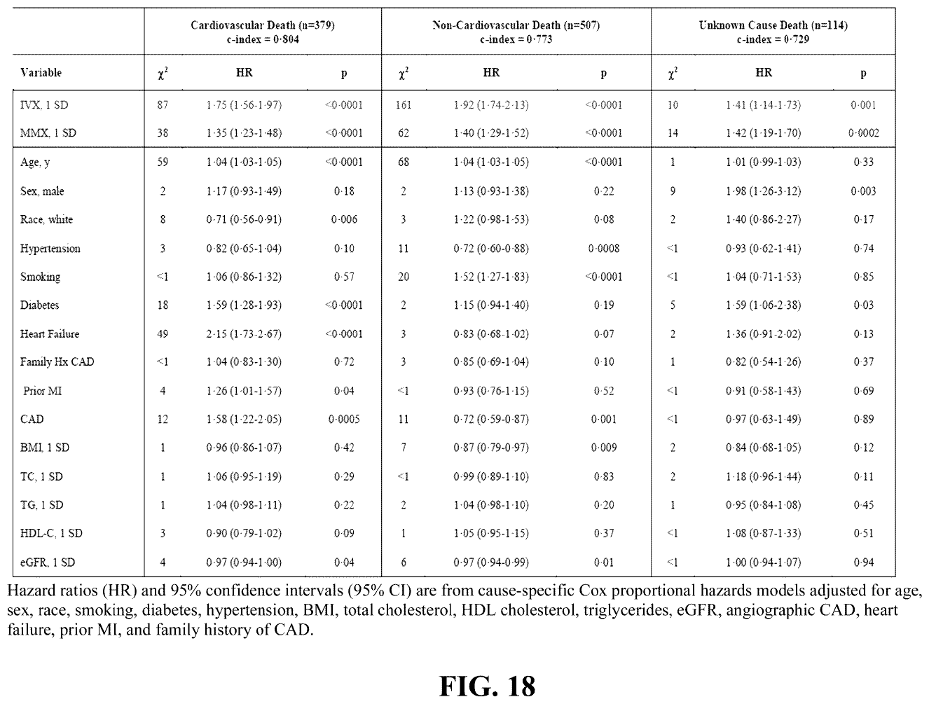

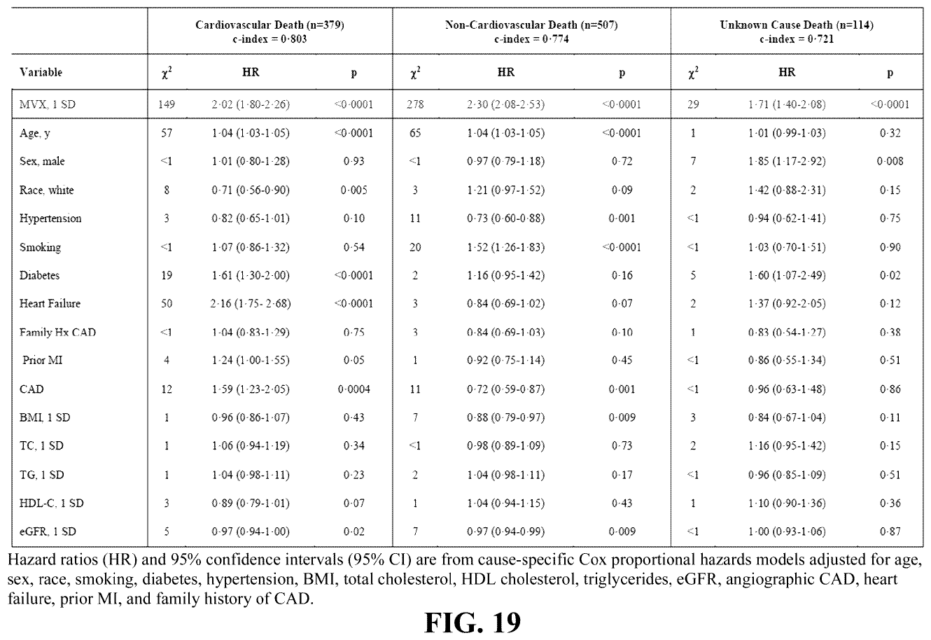

Of the 1000 deaths that occurred within 5 years in CATHGEN, 379 (38%) and 507 (51%) were from cardiovascular and non-cardiovascular causes, respectively, while 114 (11%) were of unknown cause (FIG. 18-19). Whereas some risk factors such as heart failure and diabetes predicted cardiovascular death but not non-cardiovascular death, IVX, MMX, and MVX were similarly related to mortality risk irrespective of cause (MVX HR: 2.02; 95% CI 1.80-2.26 for cardiovascular death; HR: 2.30; 95% CI 2.08-2.53 for non-cardiovascular death).

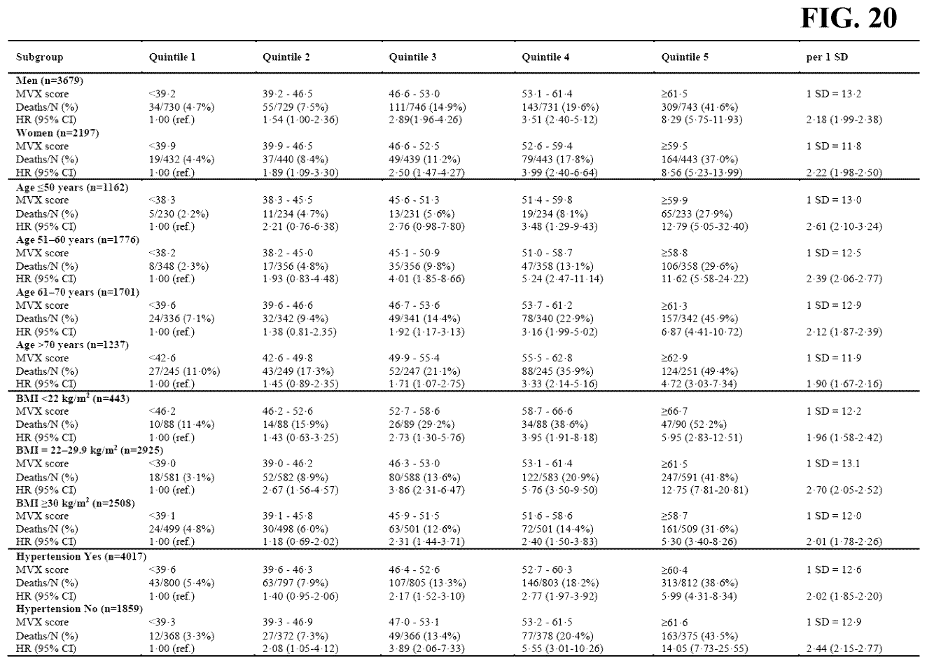

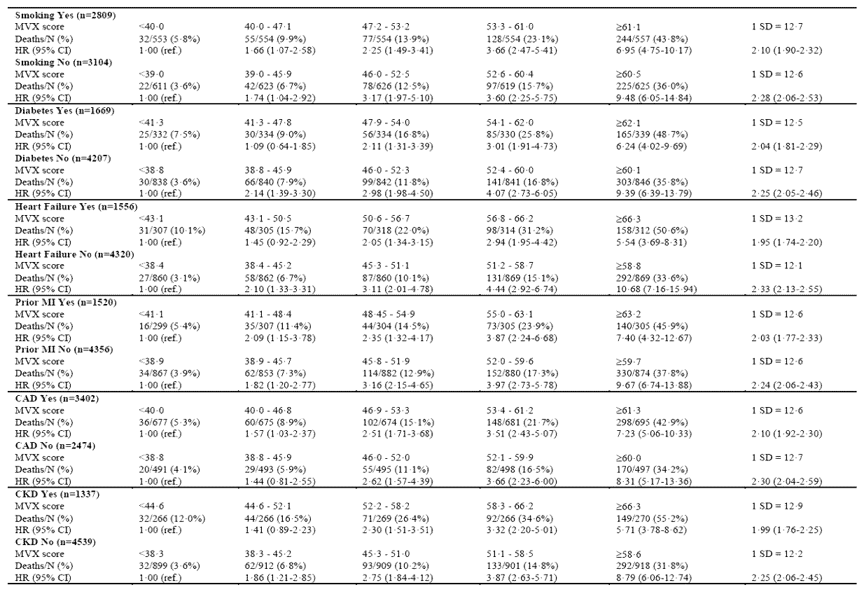

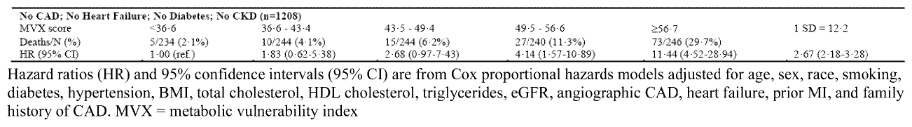

Comparative associations of MVX with all-cause mortality by sex, age, and in subgroups differing by baseline BMI, hypertension, smoking, diabetes, heart failure, prior MI, CAD, or CKD status are provided in FIG. 3 and FIG. 20. MVX associations were very similar in men (HR: 2.18; 95% CI 2.03-2.34) and women (HR: 2.22; 95% CI 1.97-2.50) and also in those with and without risk factors that impacted 5-year survival rates. This was most notably true for the roughly double mortality rates of patients with and without preexistent heart failure and CKD, and in the lowest (<22) vs highest (≥30) BMI categories. In the two lowest-risk patient subgroups, the youngest (≤50 years old) and the most “healthy” (without CAD, heart failure, diabetes, and CKD), MVX mortality associations were comparably strong (HR per 1 SD: 2.61; 95% CI 2.10-3.24 and 2.67; 95% CI 2.18-3.28, respectively).

### Conclusion

Complex and incompletely understood metabolic derangements associated with inflammation and protein-energy wasting are significant contributors to the increased mortality risk of older patients and those with chronic organ diseases stressed by the syndromes of cachexia, sarcopenia, malnutrition, and frailty. However, these wasting syndromes have uncertain relevance to cardiovascular patients or the lower-risk general population. Studies are hampered by lack of objective clinical assessment tools for these intertwined metabolic malnutrition and inflammation syndromes. This study sought to determine, in two independent cohorts of cardiac catheterization patients, the mortality risk associated with the Metabolic Vulnerability Index (MVX), a multimarker derived from six simultaneously-measured serum biomarkers plausibly linked to these dysmetabolic syndromes. The results are provocative in suggesting that survival may be more dependent on heretofore unrecognized etiologic factors distinct from those responsible for development of the diseases or vulnerabilities considered to be the “causes” of death. If borne out by future research, treating the underlying metabolic derangements of the overlap syndromes of cachexia, sarcopenia, malnutrition, and frailty with anti-inflammatory, nutritional, or alternate therapies may provide greater survival benefit than targeting conventional disease risk factors.

Clinical NMR analysis was employed to efficiently quantify six metabolites in plasma plausibly related to MMIS and showed that derived IVX, MMX, and MVX multimarker scores exhibited strong, graded associations with all-cause mortality in two large cardiac catheterization cohorts. The associations were comparably strong in men and women, younger and older and underweight and overweight patients, for death from cardiovascular and non-cardiovascular causes, and in patients with and without angiographic evidence of CAD or other comorbid conditions such as heart failure, renal dysfunction, diabetes, and hypertension. These observations and the surprising finding that MVX mortality associations were at least as strong in the two lowest-risk patient subgroups, those ≤50 years old and the phenotypically “healthy” without CAD, heart failure, CKD, or diabetes, suggest that MMIS may have general relevance to mortality risk by predisposing to greater or lesser metabolic vulnerability or resilience.

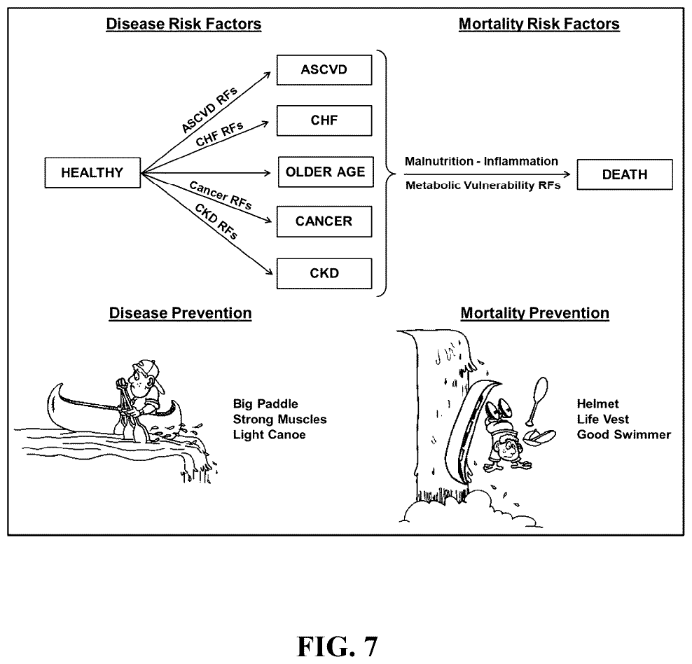

FIG. 7 conceptualizes this suggestion that there is commonality to the influence of MICS on survival irrespective of attributed cause of death. Depicted on the left are the long-term trajectories from healthy to disease states such as atherosclerotic cardiovascular disease (ASCVD) that are impacted by disease-specific risk factors (in red), the targeting of which underlies successful prevention strategies (such as lowering serum cholesterol) to reduce the risk of both fatal and nonfatal outcomes. This risk factor-targeted disease prevention strategy is likened to a canoeist employing the most pertinent tools (big paddle, etc.) to avoid being swept over the falls. Referenced on the right (in blue) is the suggestion that later in the trajectory when mortality risk is more proximal, the superimposed influence of MICS on metabolic vulnerability becomes the dominant consideration. The canoeist under these altered circumstances (high MVX score) in which survival is the paramount concern may be best served by a different risk-mitigation approach (helmet, etc.) that targets inflammation and/or nutritional status to promote metabolic resilience.

This hypothesis was advanced previously to explain “reverse epidemiology” in hemodialysis patients and other vulnerable patient subpopulations, and the failure of prevention efforts focused on conventional risk factors such as obesity, hypertension, and hypercholesterolemia to substantially improve survival. The present findings in a patient subpopulation not previously linked to MICS suggest a degree of biologic universality to the contribution of malnutrition/inflammation to mortality risk. Particularly noteworthy was the dominance of the predictive contribution of MVX to multivariable models of 5-year mortality, exceeding that of age and dwarfing the contributions of such risk factors as smoking, diabetes, and heart failure. It is not that these latter variables were unimportant in these patients; together they accounted in CATHGEN for about 55% of the mortality prediction of a Cox model that did not include MVX. Rather, adding MVX to the model improved mortality prediction so substantially that the 69% predictive contribution of MVX dwarfed those of the covariates. In contrast, risk of nonfatal MI was relatively little influenced by the MVX score and its component parts.

If MMIS plays a key role in the relatively short-term survival of cardiovascular patients, a notable consequence would be calling into question the customary use in ASCVD clinical trials of composite endpoints that combine fatal and nonfatal events. Prior critiques of composite endpoints have centered on the pitfalls of clinical interpretation and the weighting of “hard” and “soft” outcomes, but have never questioned the foundational assumption that fatal and nonfatal ASCVD events share a common etiology. They unquestionably do, but if a more potent MMIS etiology is superimposed and dominates survival in the relatively short-term, it would explain why MVX had an overriding influence on both cardiovascular and non-cardiovascular mortality, but far less on nonfatal MI. Viewed through this lens, the results of past clinical trials in which the interventions unequally affected the fatal and nonfatal components of the composite endpoint may merit reexamination.

Given the remarkably strong mortality associations of MVX and its MMX and IVX component parts, it is unclear why MICS largely has flown under the clinical radar. The likely reason is the paucity of clinically-accessible serum markers specific to the detection of coexistent protein-energy malnutrition/wasting and inflammation. The challenge is the inherent complexity of the overlap syndromes, with uncertainty as to which of the intertwined metabolic stresses are causes versus manifestations. Included among these are chronic inflammation, endocrine disorders, oxidative stress, protein hypercatabolism, acidemia, muscle anabolic resistance, defective mTOR signaling, elevated resting energy expenditure, and endothelial dysfunction. In light of this complexity, it is not surprising that a multi-marker index that aggregates six simultaneously-measured metabolites reflecting different aspects of the syndrome might have advantages over serum albumin and CRP, the usual clinical markers. The splitting of MVX into IVX and WINIX component parts served the potential future clinical purpose of treating MMIS-related mortality risk differentially depending on the reason(s) underlying MVX elevation. The arbitrary nature of ascribing IVX and MMX respectively to “inflammatory” and “metabolic malnutrition” etiologies given their apparent synergism and uncertainty about how branched-chain amino acids and citrate, in particular, are mechanistically involved, warrants recognition.

The major influence of S-HDLP on IVX and MVX scores is intriguing. Understanding how this small HDL subspecies is involved with MICS may help determine which aspects of HDL multifunctionality impact longevity and metabolic resilience. Current perceptions of the clinical importance (or lack thereof) of HDL are based almost exclusively on studies of just one HDL biomarker, HDL cholesterol (HDL-C). Since there is far more cholesterol in large versus smaller HDL particle subpopulations, clinical associations of HDL-C can misinform about the protective role(s) played by small HDL particles. The present study offers a telling example: HDL-C added to a multivariable model for mortality in CATHGEN did not increase the c-index at all, whereas addition instead of S-HDLP increased the c-index substantially. Concentrations of larger-size HDL had absolutely no association with mortality. This sharp demarcation of apparent HDL biological activity with particle size is consistent with evidence that certain HDL functional activities such as anti-oxidation, anti-inflammation, and anti-infection are mediated by proteins and/or lipid species that reside preferentially or exclusively on small-size HDL particle subpopulations.

It is too early to anticipate how MVX and its component MMX and IVX indices might ultimately be employed clinically, but several possibilities invite future study. MVX has clear mortality prognostic value, but it is not known if lowering MVX by anti-inflammatory, nutritional, or alternate therapies would extend survival. At present, the best clinical fit for MVX may be to complement or extend the “disease burden/inflammatory condition” etiologic criterion used to diagnose and grade the severity of malnutrition as it relates to the syndromes of cachexia, sarcopenia, and frailty. In this context, MVX could furnish a simple, quantitative, and objective measure of the metabolic dysfunction(s) impacting survival for which there is an unmet need, both for prognosis and to aid evaluation of the efficacy and safety of therapeutic interventions. Regarding real-world clinical practicality, MVX, IVX, and MMX scores are calculated using data from the same NMR LipoProfile “scan” currently deployed in the US for routine patient testing for cardiometabolic risk via simultaneous assessment of the lipid panel, apolipoprotein B, GlycA, and LP-IR insulin resistance score. Since the analysis utilizes no assay-specific reagents or other consumables, the MVX indices could be produced at little or no incremental analytic cost.

Overall, the MVX score, an aggregate of 6 NMR-measured biomarkers reflective of different aspects of malnutrition-inflammation syndrome, dominated the contributions made by standard cardiovascular risk factors and comorbidities to predictive models of 5-year mortality in 2 large cohorts of cardiac catheterization patients. While speculative, these results suggest that fatal and nonfatal outcomes may be influenced by different etiologies, with improved survival potentially achievable using therapies targeting one or more components of MMIS. MVX, measured in high and low-risk subjects for a cardiovascular event, made a dominant contribution to mortality prediction in cardiovascular patients overall and in relatively low-risk subgroups without preexisting disease, suggesting metabolic malnutrition-inflammation syndromes may play a more universal etiologic role influencing survival than previously thought.

The foregoing of illustrative of the present disclosure and is not to be construed as limiting thereof Although a few exemplary embodiments of this disclosure have been described, those skilled in the art will readily appreciate that many modifications are possible in the exemplary embodiments without materially departing from the novel teachings and advantages of this disclosure. Accordingly, all such modifications are intended to be included within the scope of this disclosure as defined in the claims. In the claims, means-plus-function clauses, where used, are intended to cover the structures described herein as performing the recited function and not only structural equivalents but also equivalent structures. Therefore, it is to be understood that the foregoing is illustrative of the present disclosure and is not to be construed as limited to the specific embodiments disclosed, and that modifications to the disclosed embodiments, as well as other embodiments, are intended to be included within the scope of the appended claims.

What is claimed is:

1. A method for monitoring a subject's risk of metabolic malnutrition inflammation syndrome comprising:
(a) obtaining a sample of blood, serum, or plasma from the subject;
(b) measuring at least two biomarkers simultaneously from the sample;
(c) generating a metabolic vulnerability index (MVX) value from the at least two simultaneously measured biomarkers;
(d) determining the subject's relative risk for premature death based on at least the MVX value;
(e) repeating steps (a)-(d) at a later time point; and
(f) evaluating the subject's relative risk for premature death and the MVX value over time.

2. The method of claim 1, wherein the at least two biomarkers comprise two or more of GlycA, at least one of a high density lipoprotein particle (HDLP) subclass, citrate, and a branched chain amino acid (BCAA).

3. The method of claim 2, wherein the HDLP subclass is small HDLP (S-HDLP).

4. The method of claim 2, wherein the BCAA is at least one of leucine, isoleucine, or valine.

5. The method of claim 1, wherein the MVX value is sex-specific and comprises a sex-specific inflammation vulnerability index (IVX) and a sex-specific metabolic malnutrition index (MMX).

6. The method of claim 1, wherein the MVX value is sex-specific for females and is determined using the following model:
MVX_{F}=(IVX_{F}*2.27278)+(lnMMX_{F}*12.13511)+(IVX_{F}*lnMMXF)*−1.09312.

7. The method of claim 1, wherein the MVX value is sex-specific for males is determined using the following model:
MVX_{M}=(IVX_{M}*3.54601)+(lnMMX_{M}*14.41428)+(IVX_{F}*lnMMX_{M})*−1.43438

8. The method of claim 5, wherein the measurement of the at least one of each BCAA and citrate, are used to generate the sex-specific metabolic malnutrition index (MMX) value.

9. The method of claim 5, wherein the measurement of the at least one HDLP subclass and GlycA are used to generate the sex-specific inflammation vulnerability index (IVX).

10. The method of claim 8, wherein the sex-specific MMX value for females is determined using the following model:
MMX_{F}=((4+(Leu*−0.03142)+(Leu²*0.0000893))*0.353)+((7+(Val*−0.03362)+(Val²*0.0000689))*0.684)+(Ileu*0.00332)+((1+(Citr*−0.0072)+(Citr²*0.0000573))*0.7135).

11. The method of claim 8, wherein the sex-specific MMX value for males is determined using the following model: f
ff MMXₘ=((4+(Leu*−0.01594)+(Leu²*0.0000291))*1.076)+((7+(Val*−0.0239)+(Val²*0.00005))*0.414)+(Ileu*0.01265)+((1+(Citr*0.00906)+(Citr²*−0.0000126))*0.5881).

12. The method of claim 9, wherein the sex-specific IVX value for females is determined using the following model:
IVX_{F}=9+(GlycA*−0.000187)+(S-HDLP*−0.3585)+((GlycA*S-HDLP)*0.000348.

13. The method of claim 9, wherein the sex-specific IVX value for males is determined using the following model: IVX_{M}=9+(GlycA*−0.00437)+(S-HDLP*−0.52307)+((GlycA*S-HDLP)*0.000817).

14. The method of claim 1, wherein the MVX value is sex-specific and is determined in a subject deemed to be at low-risk for a cardiovascular event or in a subject deemed to be at high-risk for a cardiovascular event.

15. The method of claim 1, wherein the measuring is performed by Nuclear Magnetic Resonance (NMR) spectrometry.

16. The method of claim 1, wherein the metabolic vulnerability index (MVX) can provide short (1 year) to long (12 year) term risk of premature death risk assessments, and wherein evaluating the subject over time comprises following up with the subject after 12 years, after 10 years, and/or every 6 years, every 5 years, every 4 years, every 3 years, every 2 years, or every 1 years.

17. A system comprising:
an NMR spectrometer configured to simultaneously acquire an NMR spectrum and/or spectra of two or more biomarkers from a blood, serum, or plasma sample from a subject; and
a processor to determine a metabolic vulnerability index (MVX) value based on the NMR spectrum and/or spectra of the two or more biomarkers, wherein the processor comprises or communicates with a memory.

18. The system of claim 17, wherein the processor is configured to determine a sex-specific inflammation vulnerability index (IVX) and a sex-specific metabolic malnutrition index (MMX).

19. The system of claim 17, wherein the NMR spectrum and/or spectra of the at least two biomarkers comprises two or more of a signal for GlycA biomarker, at least one signal for at least one high density lipoprotein particle (HDLP) subclass biomarker, at least one signal for at least one branched chain amino acid (BCAA) biomarker, and at least one signal for a citrate biomarker.

20. The system of claim 19, wherein the at least one HDLP class is a small HDLP (S-HDLP) class, and wherein the wherein the BCAA is at least one of leucine, isoleucine, or valine.
